(19) United States
(12) Patent Application Publication
(10) Pub. No.: US 2005/0176696 A1
(43) Pub. Date: Aug. 11, 2005

(54) Synergistic anit-cancer compounds

(75) Inventors: Robert T. Dorr, Tucson, AZ (US); David S. Alberts, Tucson, AZ (US); Evan M. Hersh, Phoenix, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of Arizona, Phoenix, AZ (US)

(21) Appl. No.: 11/007,988

(22) Filed: Dec. 8, 2004

Related U.S. Application Data
(60) Provisional application No. 60/528,181, filed on Dec. 8, 2003.

(51) Int. Cl. A61K 31/396

(52) U.S. Cl. 514/183

(57) ABSTRACT

The present invention provides compositions useful in treating cancer. The compositions include a synergistic combination of an antineoplastic thiol-binding mitochondrial oxidant with an antineoplastic nucleic acid binding agent, an antineoplastic antimetabolite base analog, or docetaxel. Also provided are methods of assaying the synergistic effects of the combinations and methods of treating cancer using the synergistic combinations.

### CROSS-REFERENCE TO RELATED APPLICATIONS

This application claims priority to U.S. provisional patent application number 60/528,181, filed on Dec. 8, 2003, the disclosure of which is incorporated herein by reference in its entirety for all purposes.

### STATEMENT REGARDING FEDERALLY SPONSORED RESEARCH

This invention was made with government support under CA 17094 awarded by the National Cancer Institute, National Institutes of Health. The government has certain rights in the invention.

### FIELD OF THE INVENTION

The present invention relates to methods and compositions for treating cancer using a synergistic combination of an antineoplastic thiol-binding mitochondrial oxidant and a second antineoplastic agent selected from an antineoplastic nucleic acid binding agent, an antineoplastic antimetabolite base analog, and docetaxel.

### BACKGROUND OF THE INVENTION

It is difficult to predict the effect of many combination therapies. For example, some drugs interact with each other to reduce the therapeutic effectiveness or cause undesired side-effects. These drugs are typically categorized as having an antagonistic effect. Other drug combinations manifest their therapeutic effectiveness as the sum of individual drugs. These combinations are categorized as having an additive effect. Still other drug combinations result in a therapeutic index that is greater than the sum of individual drugs. These are categorized as having a synergistic effect.

Combination therapies having a synergistic effect are highly desirable for many reasons. For example, each component in the synergistic combination therapy can be used in an amount lower than the therapeutic amount of each individual drug in monotherapy (i.e., single drug administration). Moreover, the risk and/or the severity of side-effects can be reduced significantly by reducing the amount of each drug. Furthermore, combination therapy may significantly increase the overall effectiveness of treatment. Unfortunately, however, finding combinations of drugs with synergistic effect is largely empirical.

Synergistic actions of combination therapy are particularly useful in treatments where the side-effects are extreme or severe and/or where the efficacy of monotherapy is less than desirable. For example, cancer treatment often results in nausea, vomiting, bone marrow suppression, and other severe discomfort to the patient. Similarly, treating viral infections, such as HIV infection, also results in one or more of these types of side-effects. Furthermore, the efficacy rate of cancer or HIV infection treatment is less than ideal.

In addition, development of resistance has recently become a major concern in the treatment of viral infections, such as HIV and HBV infections, as well as existing chemotherapies. Resistance usually occurs when the drugs being used are not potent enough to completely stop virus replication. If the virus can reproduce at all in the presence of drugs, it has the opportunity to mutate until it finds one that allows it to reproduce in spite of the drugs. Once a mutation occurs, it then grows unchecked and soon is the dominant strain of the virus in the individual. The drug becomes progressively weaker against the new strain. There is also increasing concern about cross-resistance. Cross-resistance occurs when mutations causing resistance to one drug also cause resistance to another. Several studies have shown that combining two drugs delays the development of resistance to one or both drugs compared to when either drug is used alone. Other studies suggest that three-drug combinations extend this benefit even further. As a result, it is believed that the best way of preventing, or at least delaying resistance is to use multi-drug combination therapies.

While some combination therapies are currently available for treating cancer and viral infections, there still is a need for additional combination therapies for cancer and viral infections. The present invention solves these and other problems.

### SUMMARY OF THE INVENTION

It has been discovered that, surprisingly, the combination of an antineoplastic thiol-binding mitochondrial oxidant with an antineoplastic nucleic acid binding agent, an antineoplastic antimetabolite base analog, or docetaxel, is synergistic when used to treat individuals with cancer.

In a first aspect, the present invention provides a method for treating cancer in a human in need of such a treatment. The method includes administering to the patient a therapeutically effective amount of a composition. The composition includes an antineoplastic thiol-binding mitochondrial oxidant and an antineoplastic nucleic acid binding agent. The amount provides a synergistic therapeutic cytotoxic effect.

In another aspect, the present invention provides a method for treating cancer in a human in need of such a treatment. The method includes administering to the patient a therapeutically effective amount of a composition. The composition includes an antineoplastic thiol-binding mitochondrial oxidant and an antineoplastic antimetabolite base analog. The amount provides a synergistic therapeutic cytotoxic effect.

In another aspect, the present invention provides a method for treating cancer in a human in need of such a treatment. The method includes administering to the patient a therapeutically effective amount of a composition. The composition includes an antineoplastic thiol-binding mitochondrial oxidant and an docetaxel. The amount provides a synergistic therapeutic cytotoxic effect.

### BRIEF DESCRIPTION OF THE DRAWINGS

FIG. 1 is a representation of combination index data for imexon in combination with cisplatin, dacarbazine (DTIC), melphalan or taxotere in A375 cells.

FIG. 2 is a representation of combination index data for imexon in combination with cisplatin, dacarbazine (DTIC), melphalan or taxotere in 8226/s cells.

FIG. 3 is a representation of combination index data for imexon in combination with cytarabine, 5-fluorouracil, or gemcitabine in A375 cells.

FIG. 4 is a representation of combination index data for imexon in combination with cytarabine, 5-fluorouracil, or gemcitabine in 8226/s cells.

FIG. 5 is a representation of combination index data for imexon in combination with methotrexate or doxorubicin in A375 cells.

FIG. 6 is a representation of combination index data for imexon in combination with dexamethasone, doxorubicin, methotrexate, or paclitaxel in 8226/s cells.

FIG. 7 is a representation of combination index data for imexon in combination with dexamethasone, paclitaxel, or vinorelbine in A375 cells.

FIG. 8 is a representation of combination index data for imexon in combination with vinorelbine in 8226/s cells.

FIG. 9 is a representation of the anti-pancreatic tumor effects of imexon in combination with gemcitabine in mice.

FIG. 10 is a representation of the anti-leukemia effects of imexon in combination with cytarabine in mice.

FIG. 11 is a representation of the antagonistic effect of imexon in combination with the topoisomerase inhibitor irinotecan in Human Multiple Myeloma Cells (8226/s) in vitro.

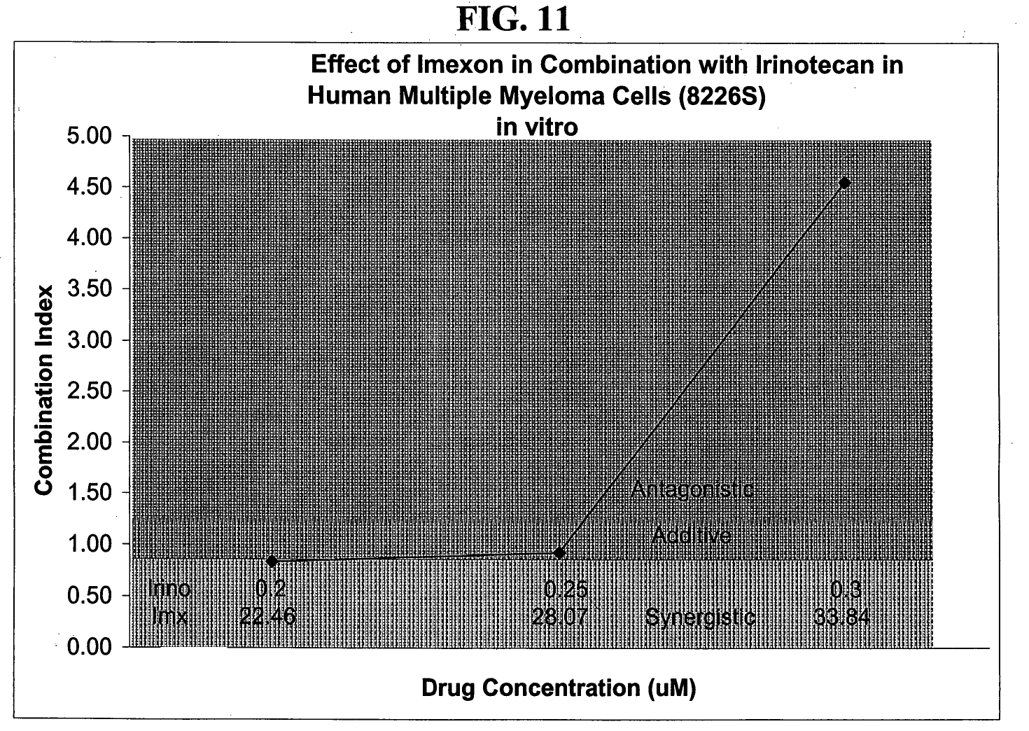

### DETAILED DESCRIPTION OF THE INVENTION

I. Definitions

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “antineoplastic” means inhibiting or preventing the growth of cancer. “Inhibiting or preventing the growth of cancer” includes reducing the growth of cancer relative to the absence of a given therapy or treatment. Cytotoxic assays useful for determining whether a compound is antineoplastic are discussed below (see Assays for Testing the Anticancer Synergistic Activity of a Combination of an Antineoplastic Thiol-binding Mitochondrial Oxidant and a Second Antineoplastic Agent).

As used herein “combination therapy” or “adjunct therapy” means that the patient in need of the drug is treated or given another drug for the disease in conjunction with antineoplastic thiol-binding mitochondrial oxidant. This combination therapy can be sequential therapy where the patient is treated first with one drug and then the other or the two drugs are given simultaneously.

“Imexon” refers to an unsubstituted 4-imino-1,3-diazabicyclo[3.1.0]-hexan-2-one, or a pharmaceutically acceptable salt or a solvate thereof.

“Patient” refers to a mammalian subject, including human.

A “synergistic therapeutic cytotoxic effect,” as used herein, means that a given combination of at least 2 compounds exhibits synergy when tested in a cytotoxic assay (see Assays for Testing the Anticancer Synergistic Activity of a Combination of an Antineoplastic Thiol-binding Mitochondrial Oxidant and a Second Antineoplastic Agent, below). Synergy is assessed using the median-effect principle (Chou, et al., Adv Enzyme Regul 22:27-55 (1984)). This method is based on Michaelis-Menton kinetics and reduces combination effects to a numeric indicator, the combination index (C.I.). Where the combination index is less than 1, synergism is indicated. Where the combination index is equal to 1, summation (also commonly referred to as additivity) is indicated. Where the combination index is greater than 1, antagonism is indicated.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3 -piperidinyl, 4-morpholinyl, 3-morpholinyl, 3-thiomorpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, alkyl, heteroalkyl, aryl and heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR—′, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

As used herein, “nucleic acid” means either DNA, RNA, single-stranded, double-stranded, or more highly aggregated hybridization motifs, and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

II. Synergistic Compositions Useful in Treating Cancer

In one aspect, the present invention provides novel compositions useful in treating cancer. The compositions include an antineoplastic thiol-binding mitochondrial oxidant and a second antineoplastic agent selected from antineoplastic nucleic acid binding agent, antineoplastic antimetabolite base analog, and docetaxel. It has been discovered that, surprisingly, the combination of the antineoplastic thiol-binding mitochondrial oxidant and the second antineoplastic agent exhibit a synergistic therapeutic cytotoxic effect.

The compositions of the current invention are useful in treating a wide variety of cancers, including carcinomas, sarcomas, and other forms of cancer. Exemplary cancers include multiple myeloma, a β-lymphocyte plasmacytoma, ovarian cancer (e.g. advanced stage ovarian epithelial cell cancer), melanoma (e.g. metastatic melanoma, leukemia (including leukemias of lymphoid and nonlymphoid origin), colon cancer (e.g. metastatic colon cancer), breast cancer, lung cancer (e.g. and metastatic lung cnacer), and pancreatic cancer (including neoplasms of the endocrine and exocrine pancreas). Exemplary endoneoplastic pancreatic disorders include nonfunctional endocrine neoplasm, somatostatinoma, glucagonoma, VIPoma, gastrinoma, and insulinoma.

A. Antineoplastic Thiol-Binding Mitochondrial Oxidants

Antineoplastic thiol-binding mitochondrial oxidants of the present invention are those compounds that inhibit or prevent the growth of cancer, are capable of binding a thiol moiety on a thiol-containing molecule, and promote oxidative stress and disrupt cellular mitochondrial membrane potential. An antineoplastic thiol-binding mitochondrial oxidant typically induces gross alterations in mitochondrial ultrastructure (such as swelling), while inducing little or no alterations to other cellular organelles. Alterations in the mitochondrial ultrastructure is typically caused by induction of oxidative stress to mitochondrial biomolecules, such as mitochondrial DNA. In addition to oxidative damage to mitochondrial DNA and changes in mitochondrial morphology, antineoplastic thiol-binding mitochondrial oxidants will typically cause a buildup of reactive oxygen species (ROS) in addition to perturbations in mitochondrial membrane potential, leading to cytchrome c release, activation of caspases 3, 8, and 9, and induction of apoptosis.

In some embodiments, the antineoplastic thiol-binding mitochondrial oxidant inhibits or reduces activity of a ribonucleotide reductase inhibitor (relative to the activity in the absence of an antineoplastic thiol-binding mitochondrial oxidant). In other embodiments, the antineoplastic thiol-binding mitochondrial oxidant does not alkylate DNA. In another embodiment, the antineoplastic thiol-binding mitochondrial oxidant does not react with the ε-amino group of lysine.

Techniques for measuring characteristics of antineoplastic thiol-binding mitochondrial oxidants are discussed below and disclosed in detail in Dvorakova et al., Neoplasia 97: 3544-3551(2001), Dvorakova et al., Biochemical Pharmacology 60: 749-758 (2000), Dvorakova et al., Anti-Cancer Drugs 13: 1031-1042 (2002), Dvorakova et al., Molecular Cancer Therapeutics 1: 185-195 (2002), and Iyengar et al., J. Med. Chem. 47, 218-223 (2004).

In an exemplary embodiment, the antineoplastic thiol-binding mitochondrial oxidant includes an aziridine ring (e.g. the compounds of Formulae (I), (II), and (III)). The aziridine ring enables the antineoplastic thiol-binding mitochondrial oxidant to bind cellular thiols, such as glutathione S transferase (GSH) and cysteine residues within cellular proteins. As a consequence of depleting cellular thiols such as cysteine and GSH, tumor cells become highly susceptible to oxidation.

In an exemplary embodiment, the antineoplastic thiol-binding mitochondrial oxidant having an aziridine ring is a substituted or unsubstituted aziridine-1-carbaoxamide having the formula:

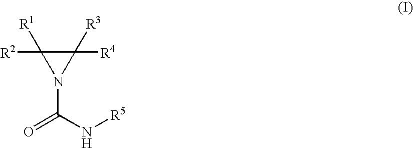

In Formula (I), R¹, R², R³, R⁴ and R⁵ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R⁴ and R⁵ are optionally joined together to form a substituted or unsubstituted 5 to 7 membered ring.

In a related embodiment, R⁴ is cyano, CO₂R^{4A}, or CONR^{4B}R^{4C}. R^{4A} is selected from substituted or unsubstituted alkyl, substituted or unsubstituted cycloalkyl, and substituted or unsubstituted aryl. R^{4B} is hydrogen, or substituted or unsubstituted alkyl. R^{4C} is hydrogen substituted or unsubstituted alkyl, substituted or unsubstituted heterocycloalkyl, or substituted or unsubstituted aryl. In a further related embodiment, R⁴ is cyano.

In another related embodiment, R¹, R² and R³ are independently selected from hydrogen, substituted or unsubstituted (C₁-C₆)alkyl, substituted or unsubstituted 2 to 6 membered heteroalkyl, substituted or unsubstituted (C₁-C₆)cycloalkyl, substituted or unsubstituted 5 to 7 membered heterocycloalkyl, substituted or unsubstituted aryl, and substituted or unsubstituted heteroaryl. R⁴ is cyano, unsubstituted carboxamide or unsubstituted carboxylic acid ester. R⁵ hydrogen or substituted or unsubstituted (C₁-C₄) alkyl. R⁶ is substituted or unsubstituted (C₁-C₈) alkyl, a substituted or unsubstituted 5 to 7 membered heterocycloalkyl, or a substituted or unsubstituted aryl.

In another related embodiment, R⁴ and R⁵ are joined together to form a substituted 5 membered ring. In a further related embodiment, the compound of Formula (I) is imexon. In an exemplary embodiment where imexon is the antineoplastic thiol-binding mitochondrial oxidant, the concentration of imexon in the composition is at least 0.5 μg/ml. In another exemplary embodiment, the concentration of imexon in the composition is at least 1.0 μg/ml. In another exemplary embodiment, the concentration of imexon in the composition is between 1.0 μg/ml and 500 μg/ml.

In another exemplary embodiment, the antineoplastic thiol-binding mitochondrial oxidant is selected from a substituted or unsubstituted aziridine-1-carboxamide and a substituted or unsubstituted 4-imino-1,3-diazobicyclo[3.1.0]-hexane-2-one. Aziridine-1-carboxamides and cyclic derivatives thereof useful in the present invention are discussed in detail in U.S. Pat. No. 6,297,230 and U.S. Pat. No. 6,476,236, which are assigned to the same assignee as the present application and are herein incorporated by reference in their entirety for all purposes.

Useful substituted or unsubstituted 4-imino-1,3-diazobicyclo[3.1.0]-hexane-2-ones may have the formula:

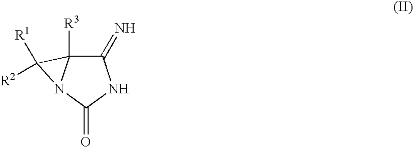

In Formula (II), R¹, R² and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R¹, R² and R³ are independently selected from hydrogen, substituted or unsubstituted (C₁-C₆)alkyl, substituted or unsubstituted 2 to 6 membered heteroalkyl, substituted or unsubstituted (C₁-C₆)cycloalkyl, substituted or unsubstituted 5 to 7 membered heterocycloalkyl, substituted or unsubstituted aryl, and substituted or unsubstituted heteroaryl.

In a related embodiment, R¹, R² and R³ are independently selected from hydrogen and hydrogen, substituted or unsubstituted (C₁-C₆)alkyl.

In another related embodiment, R¹, R² and R³ are hydrogen. One of skill in the art will recognize that where R¹, R² and R³ are hydrogen, the compound of Formula I is imexon. Thus, in a related embodiment, the antineoplastic thiol-binding mitochondrial oxidant is imexon.

In an exemplary embodiment, the substituted or unsubstituted aziridine-1-carboxamide has the formula:

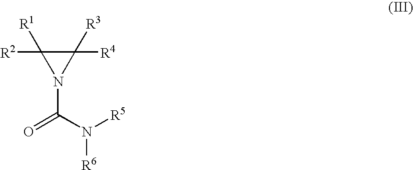

In Formula (III), R¹, R² and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R⁴ is cyano, CO₂R^{4A}, or CONR^{4B}R^{4C}. R^{4A} is selected from substituted or unsubstituted alkyl, substituted or unsubstituted cycloalkyl, and substituted or unsubstituted aryl. R^{4B} is hydrogen, or substituted or unsubstituted alkyl. R^{4C} is hydrogen substituted or unsubstituted alkyl, substituted or unsubstituted heterocycloalkyl, or substituted or unsubstituted aryl. R⁵ is hydrogen or substituted or unsubstituted alkyl. R⁶ is substituted or unsubstituted alkyl, substituted or unsubstituted heterocycloalkyl, or substituted or unsubstituted aryl.

In a related embodiment, R⁴ is cyano. Where R⁴ is cyano, the molecule may be referred to herein as a substituted or unsubstituted cyanoaziridine.

In an exemplary embodiment, R¹, R² and R³ are independently selected from hydrogen, substituted or unsubstituted (C₁-C₆)alkyl, substituted or unsubstituted 2 to 6 membered heteroalkyl, substituted or unsubstituted (C₁-C₆)cycloalkyl, substituted or unsubstituted 5 to 7 membered heterocycloalkyl, substituted or unsubstituted aryl, and substituted or unsubstituted heteroaryl. R⁴ is cyano, unsubstituted carboxamide or unsubstituted carboxylic acid ester. R⁵ hydrogen or substituted or unsubstituted (C₁-C₄) alkyl. R⁶ is substituted or unsubstituted (C₁-C₈) alkyl, a substituted or unsubstituted 5 to 7 membered heterocycloalkyl, or a substituted or unsubstituted aryl.

In a related embodiment, R¹, R² and R³ are independently selected from hydrogen and substituted or unsubstituted (C₁-C₆)alkyl. R⁴ is cyano and R⁵ is hydrogen.

B. Antineoplastic Nucleic Acid Binding Agents

In another aspect, the present invention provides a pharmaceutical composition including an antineoplastic thiol-binding mitochondrial oxidant and an antineoplastic nucleic acid binding agent. It has been discovered that, surprisingly, the combination of an antineoplastic thiol-binding mitochondrial oxidant and an antineoplastic nucleic acid binding agent exhibits a synergistic therapeutic cytotoxic effect.

Antineoplastic nucleic acid binding agents inhibit or prevent the growth of cancer and covalently attach substituted or unsubstituted alkyl groups, substituted or unsubstituted heteroalkyl, substituted or unsubstituted cycloalkyl, substituted or unsubstituted heterocycloalkyl, substituted or unsubstituted aryl, or substituted or unsubstituted heteroaryl groups to nucleophilic sites on a cellular nucleic acid. Typically, the antineoplastic nucleic acid binding agent are electrophilic species that will cause cross-linking of nucleic acid strands, abnormal base pairing, depurination, excision repair of alkylated nucleic acids, and/or nucleic acid strand breakage. Thus, antineoplastic nucleic acid binding agents may be monofunctional (one reactive group), bifunctional (two reactive groups) or polyfunctional (three or more reactive groups). Although the antineoplastic nucleic acid binding agents are not constrained by a particular mechanism of action, the N⁷, O⁶, and 2-amino nitrogen of guanine are particularly susceptible to antineoplastic nucleic acid binding agents.

Assays for determining whether a compound covalently attaches to a nucleophilic site on a cellular nucleic acid are well known in the art. A more detailed discussion of such assays are described in detail, for example in Price et al., “Chemistry of Alkylation” in Antineoplastic and Immunosuppressive Agents, Part II, Ed by Sartorelli et al., Berlin, Springer-Verlag, 1975, pp. 1-5; Johnson et al., Molec Pharmacol 3: 195 (1967); and Kohn, et al., Cancer Res 37: 1450 (1977).

In an exemplary embodiment, the antineoplastic nucleic acid binding agent covalently attaches substituted or unsubstituted alkyl groups, substituted or unsubstituted heteroalkyl, substituted or unsubstituted cycloalkyl, substituted or unsubstituted heterocycloalkyl, substituted or unsubstituted aryl, or substituted or unsubstituted heteroaryl groups to nucleophilic sites on a nucleic acid. In a further embodiment, the nucleophilic site on the nucleic acid is the N⁷, O⁶, and 2-amino nitrogen a guanine nitrogenous base.

In another exemplary embodiment, the antineoplastic nucleic acid binding agent is an antineoplastic DNA binding agent. An antineoplastic DNA binding agent covalently attaches substituted or unsubstituted alkyl groups, substituted or unsubstituted heteroalkyl, substituted or unsubstituted cycloalkyl, substituted or unsubstituted heterocycloalkyl, substituted or unsubstituted aryl, or substituted or unsubstituted heteroaryl groups to nucleophilic sites on cellular DNA.

A variety of antineoplastic nucleic acid binding agents are useful in the present invention, including, for example, antineoplastic nitrogen mustards, antineoplastic alkyl sulfonates, antineoplastic nitroso ureas, antineoplastic platinum complexes, antineoplastic imidazole carboxamides, altretamine and derivatives thereof, mitomycin C and derivatives thereof, benzoquinone-containing bindinging agents, and thiotepa and derivatives thereof. In an exemplary embodiment, the antineoplastic nucleic acid binding agent is selected from antineoplastic nitrogen mustard, antineoplastic imidazole carboxamide, and antineoplastic platinum complex. In another exemplary embodiment, the antineoplastic nucleic acid binding agent is selected from melphalan, cyclophosphamide, carmustine, mechlorethamine, thiotepa, chlorambucil, lomustine, ifosfamide, mitomycin C, cisplatin, carboplatin, oxaliplatin and dacarbazine. In another exemplary embodiment, the antineoplastic nucleic acid binding agent is selected from melphalan, carmustine, mechlorethamine, thiotepa, chlorambucil, lomustine, ifosfamide, mitomycin C, cisplatin, carboplatin, oxaliplatin and dacarbazine. Thus, in some embodiments, the antineoplastic nucleic acid binding agent is not cyclophosphamide.

Antineoplastic nitrogen mustards useful in the current invention include those compounds having chlorinated leaving groups that covalently bind to reactive groups on DNA, RNA, and/or polypeptide molecules. In an exemplary embodiment, the nitrogen mustard has the formula:
(Cl₂CH₂)₂N—R¹ (IV)

In Formula (IV), R¹ is selected from substituted or unsubstituted alkyl, substituted or unsubstituted heteroalkyl, substituted or unsubstituted, substituted or unsubstituted cycloalkyl, substituted or unsubstituted heterocycloalkyl, substituted or unsubstituted aryl, and substituted or unsubstituted heteroaryl. In a related embodiment, R¹ is selected from substituted or unsubstituted alkyl, substituted or unsubstituted aryl, and substituted or unsubstituted heterocycloalkyl. In a further related embodiment, R¹ is selected from substituted or unsubstituted (C₁-C₅) alkyl, substituted or unsubstituted phenyl, and substituted or unsubstituted cyclophosphamide. In another related embodiment, R¹ is substituted phenyl.

In another exemplary embodiment, the nitrogen mustard is selected from mechlorethamine, melphalan, cyclophosphamide, and chlorambucil and derivatives thereof. In a related embodiment, the nitrogen mustard is selected from melphalan and cyclophosphamide. In another related embodiment, the nitrogen mustard is selected from chlorambucil and melphalan.

In another exemplary embodiment, the nitrogen mustard is not cyclophosphamide.

Antineoplastic platinum complexes useful in the current invention include those compounds that form interstrand or intrastrand adducts to and/or crosslink cellular macromolecules, such as DNA. Typically, the platinum complexes include a platinum II (Pt²⁺) or platinum IV species (Pt⁴⁺)

In an exemplary embodiment, the antineoplastic platinum complex has the formula:

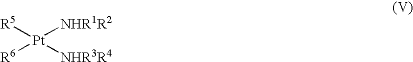

In Formula (V), R¹, R², R³, and R⁴ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R¹ and R² are optionally joined together to form a ring with the platinum to which they are attached. R⁵ is selected from halogen and OR⁷. R⁶ are independently selected from halogen and OR⁸. R⁷ and R⁸ are independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R⁷ and R⁸ are optionally joined together with the atoms to which they are attached to from a ring.

In another exemplary embodiment, the antineoplastic platinum complex is selected from cisplatin, carboplatin, oxaliplatin, and derivatives thereof. In another exemplary embodiment, the antineoplastic platinum complex is selected from cisplatin, carboplatin, and oxaliplatin. In another exemplary embodiment, the antineoplastic platinum complex is selected from cisplatin, carboplatin.

In an exemplary embodiment, the antineoplastic imidazole carboxamide has the formula:

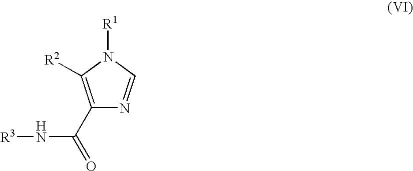

In Formula (VI), R¹, R², and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R¹ and R² may optionally be joined together to from a ring.

In a related embodiment, R² is —N═N—N—R⁴. R⁴ is a substituted or unsubstituted (C₁-C₅) alkyl or a substituted or unsubstituted (C₁-C₅) alkylene joined with R¹ to form a ring. In a further related embodiment, R³ is hydrogen.

In another exemplary embodiment, the antineoplastic imidazole carboxamide is selected from temozolomide, dacarbazine, and derivatives thereof. In another exemplary embodiment, the antineoplastic imidazole carboxamide is dacarbazine.

In another exemplary embodiment, the antineoplastic nucleic acid binding agent is selected from melphalan, cyclophosphamide, carmustine, mechlorethamine, thiotepa, chlorambucil, lomustine, ifosfamide, mitomycin C, cisplatin, carboplatin, oxaliplatin, dacarbazine, and derivatives thereof. In another exemplary embodiment, the antineoplastic nucleic acid binding agent is selected from melphalin, cisplatin and dacarbazine and derivatives thereof. In another exemplary embodiment, the antineoplastic nucleic acid binding agent is not cyclophosphamide.

Antineoplastic alkyl sulfonates of the present invention typically contain at least one electron deficient sulfonate group. Carbonium ions are rapidly formed after systemic absorption of antineoplastic alkyl sulfonates leading to alkylation of DNA.

In an exemplary embodiment, the alkyl sulfonate has the structure:

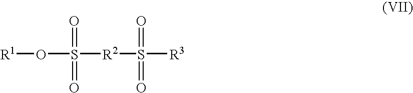

In Formula (VII), R¹ and R³ are independently selected from substituted or unsubstituted alkyl and substituted or unsubstituted heteroalkyl. R² is selected from substituted or unsubstituted alkylene and substituted or unsubstituted heteroalkylene. In a related embodiment R¹ and R³ are unsubstituted alkyl and R² is unsubstituted alkylene. In a further related embodiment, R¹ and R³ are unsubstituted (C₁-C₅) alkyl and R² is unsubstituted (C₁-C₅) alkylene.

In another embodiment, the alkyl sulfonate is busulfan or a derivative thereof. In a related embodiment, the alkyl sulfonate is busulfan.

In another exemplary embodiment, the mitomycin derivatives of the present have the formula

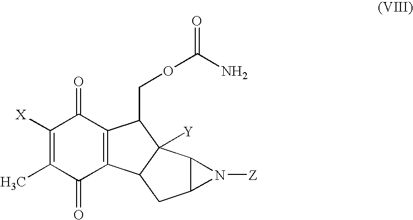

In Formula (VIII), X is selected from, ═NR¹, NHR² and OR³. R¹ is selected from substituted or unsubstituted alkyl and substituted or unsubstituted heteroalkyl. R² and R³ are independently selected from hydrogen, substituted or unsubstituted alkyl, substituted or unsubstituted heteroalkyl, and substituted or unsubstituted aryl. Y is OR³, where R³ is selected from hydrogen and substituted or unsubstituted alkyl. Z is selected from hydrogen and substituted or unsubstituted alkyl.

In a related embodiment, R¹ is a substituted or unsubstituted 2 to 5 membered heteroalkyl. In another related embodiment, R² is a hydrogen, substituted or unsubstituted 2 to 5 membered heteroalkyl and substituted or unsubstituted aryl. In another related embodiment Y is selected from —OCH₃ and —OH. In another related embodiment, Z is selected from hydrogen and —CH₃.

In another exemplary embodiment, the mitomycin derivatives include Mitomycin A, Mitomycin B, Mitomycin C, Porfiromycin, BMY-25282, BMS-181174, KW2149, and M83.

In another exemplary embodiment, benzoquinone-containing binding agents have the formula:

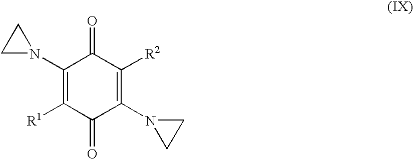

In Formula (IX), R¹ is selected from NHR³, substituted or unsubstituted alkyl, substituted or unsubstituted heteroalkyl, and substituted or unsubstituted heterocycloalkyl. R² is selected from hydrogen, NHR⁴, substituted or unsubstituted alkyl, and substituted or unsubstituted heteroalkyl. R³ and R⁴ are independently selected from substituted or unsubstituted alkyl and substituted or unsubstituted heteroalkyl.

In a related embodiment, R¹ is selected from methyl, azridinyl, and NHR³, where R³ is a substituted or unsubstituted C₁-C₅ alkyl. In a further related embodiment, R³ is CO₂CH₂CH₃ or CH₂CH₂OH.

In another exemplary embodiment, the nitroso ureas of the present invention include bis-chloroethylnitrosourea (BCNU), N-(2-chloroehtyl)-N′-(4-cylcohexyl)-N-nitrosourea (CCNU), N-(2-chloroehtyl)-N′-(4-cylcohexyl)-N-nitrosourea (methyl-CCNU), and derivatives thereof. In another exemplary embodiment, the nitrosourea had the formula:

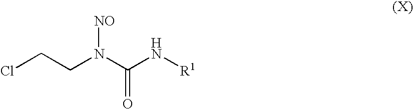

In Formula (X), R′ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In a related embodiment, R′ is selected from substituted or unsubstituted alkyl, and substituted or unsubstituted cycloalkyl.

C. Antineoplastic Antimetabolite Base Analogs

In another aspect, the present invention provides a pharmaceutical composition including an antineoplastic thiol-binding mitochondrial oxidant and an antineoplastic antimetabolite base analog. It has been discovered that, surprisingly, the combination of an antineoplastic thiol-binding mitochondrial oxidant and an antineoplastic antimetabolite base analog exhibit a synergistic therapeutic cytotoxic effect.

Antineoplastic antimetabolite base analogs inhibit or prevent the growth of cancer and disrupt cellular nucleic acid synthesis by inhibiting cellular nucleic acid synthesis enzymes. Inhibition of cellular nucleic acid synthesis enzymes is typically accomplished by mimicking the structure of natural nucleosides, nucleotides, and/or nitrogenous bases (i.e. adenine, guanine, uracil, cytosine, or thymine). Thus, antineoplastic antimetabolite base analogs of the present invention include analogs of adenine, guanine, uracil, cytosine, or thymine nucleotides, nucleosides and/or nitrogenous bases.

Assays for determining whether a compound inhibits cellular nucleic acid enzymes are well known in the art. A more detailed discussion of such assays are described in detail, for example in Hitchings et al., “Mechanisms of action of purine and pyrimidine analogs” in Cancer Chemotherapy, Basic and Clinical Applications, ed. by Brodsky, et al, New York, Grune and Stratton, 1967, pp: 26-36; Santi, et al., Biochemistry 13: 471 (1974); Waqar et al., Biochem. Journal, 121: 803 (1971); and Huang et al., Cancer Res 51: 6110-6117, (1991).

In an exemplary embodiment, the antineoplastic antimetabolite base analog has the formula:

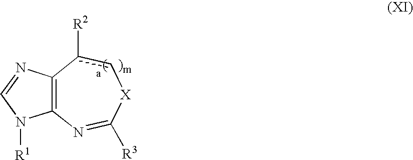

In Formula (XI), R¹ is selected from hydrogen, substituted ribose and substituted deoxyribose. R² is selected from hydrogen, halogen, —SH, —NH₂, —OH, ═O, and —SR⁴. R⁴ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R³ is selected from hydrogen, halogen, —SH, —NH₂, and —OH. The dashed line a is single bond or double bond. X is selected from ═N— or —NH—, where if a is a double bond and m is 0 then X is ═N—, and if m is 1 then X is —NH—. The symbol m is the integer 0 or 1. Where R² is ═O or m is 1, the dashed line a is a single bond.

In a related embodiment, R² is selected from —NH₂, —OH, —SH and —SR⁴.

In another related embodiment, R⁴ is selected from substituted or unsubstituted cycloalkyl, substituted or unsubstituted heterocycloalkyl, substituted or unsubstituted aryl, and substituted or unsubstituted heteroaryl. In another related embodiment, R⁴ is selected from substituted or unsubstituted heterocycloalkyl and substituted or unsubstituted heteroaryl.

In another related embodiment, R³ is selected from hydrogen, F, Cl, and —NH₂,

In another related embodiment, R¹ is selected from substituted ribose and substituted deoxyribose. The substituted ribose and substituted deoxyribose may be identical to the ribose and deoxyribose rings found in cellular DNA or RNA. Alternatively, the substituted ribose and substituted deoxyribose may be analogs of the ribose and deoxyribose rings found in cellular DNA or RNA. For example, the hydroxyl attached to the 2′C of a ribose may be an α-OH or β-OH. The 5′C may be attached to a hydroxyl, a phosphoester, a phosphodiester, or a phosphotriester moiety, or phosphoester derivatives thereof (such as phosphothioesters).

In another related embodiment, m is 0.

Thus, in another exemplary embodiment, the antineoplastic antimetabolite base analog has the formula:

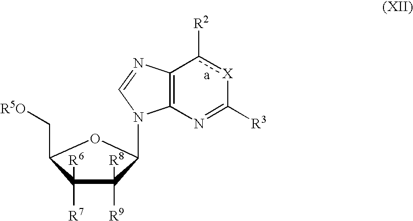

In Formula (XII), R² and R³ are as defined in Formula (XI) above. R⁶, R⁷, R⁸, and R⁹ are independently selected from hydrogen, halogen, —OH, and OR¹⁰. R¹⁰ is selected from substituted or unsubstituted alkyl, and substituted or unsubstituted heteroalkyl. R⁵ is selected from substituted or unsubstituted alkyl and —P(X′)O₂—R¹¹. R¹¹ is selected from substituted or unsubstituted alkyl, substituted or unsubstituted heterocycloalkyl, —P(XI)O₂ and —P(X²)O—P(X¹)O₂. X¹, X² and X³ are independently selected from O and S. The dashed line a is single bond or double bond. Where R² is ═O, the dashed line a is a single bond. X is selected from ═N— and —NH—, wherein if a is a double bond then X is ═N— and if a is a single bond then X is —NH—.

In a related embodiment, R⁶, R⁷, R⁸, and R⁹ are independently selected from hydrogen, F, —OH, and OR¹⁰.

In another exemplary embodiment, the antineoplastic antimetabolite base analog has the formula:

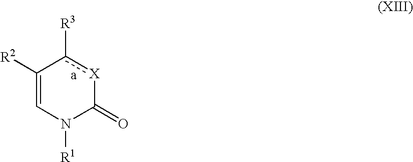

In Formula (XIII) R¹ is selected from hydrogen, substituted ribose and substituted deoxyribose. R² is selected from hydrogen, halogen, and substituted or unsubstituted alkyl. R³ is selected from hydrogen, ═O, NH₂, NH₂.HCl, and substituted or unsubstituted alkyl. The dashed line a is single bond or double bond. Where R³ is ═O, the dashed line a is a single bond. X is selected from ═N— and —NH—, wherein if a is a double bond then X is ═N— and if a is a single bond then X is —NH—.

In a related embodiment, R² is selected from hydrogen, F, and substituted or unsubstituted (C₁-C₅) alkyl. In another related embodiment, R² is selected from hydrogen, F, and unsubstituted (C₁-C₅) alkyl.

In another related embodiment, R¹ is selected from substituted ribose and substituted deoxyribose. The substituted ribose and substituted deoxyribose may be identical to the ribose and deoxyribose rings found in cellular DNA or RNA. Alternatively, the substituted ribose and substituted deoxyribose may be analogs of the ribose and deoxyribose rings found in cellular DNA or RNA. For example, the hydroxyl attached to the 2′C of a ribose may be an α-OH or β-OH. The 5′C may be attached to a hydroxyl, a phosphoester, a phosphodiester, or a phosphotriester moiety, or phosphoester derivatives thereof (such as phosphothioesters).

Thus, in another exemplary embodiment, the antineoplastic antimetabolite base analog has the formula:

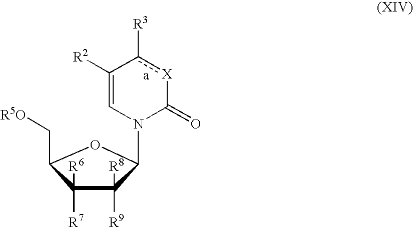

In Formula (XIV), R², R³, X and a are as defined above in Formula (XIII). R⁵, R⁶, R⁷, R⁸, and R⁹ are as defined above in Formula (XII).

In another exemplary embodiment, the antineoplastic antimetabolite base analog is selected from mercaptopurine, thioguanine, azathioprine, fludarabine, cladribine, pentostatin, fluorouracil, cytarabine, capecitabine, gemcitabine, floxuridine, and derivatives thereof In another exemplary embodiment, the antineoplastic antimetabolite base analog is selected from mercaptopurine, thioguanine, azathioprine, fludarabine, cladribine, pentostatin, fluorouracil, cytarabine, capecitabine, gemcitabine, and floxuridine. In another exemplary embodiment, the antineoplastic antimetabolite base analog is selected from 5-fluorouracil, cytarabine, and gemcitabine.

D. Docetaxel

In another aspect, the present invention provides a pharmaceutical composition including an antineoplastic thiol-binding mitochondrial oxidant and docetaxel (also referred to herein by its trade name, Taxotere®). It has been discovered that, surprisingly, the combination of antineoplastic thiol-binding mitochondrial oxidant and docetaxel exhibit a synergistic therapeutic cytotoxic effect.

III. Assays for Testing the Anticancer Synergistic Activity of a Combination of an Antineoplastic Thiol-binding Mitochondrial Oxidant and a Second Antineoplastic Agent

In another aspect, the present invention provides assays to determine whether a combination of an antineoplastic thiol-binding mitochondrial oxidant and a second antineoplastic agent has a synergistic therapeutic cytotoxic effect. As defined above, a “synergistic therapeutic cytotoxic effect” means that a given combination of at least 2 compounds exhibits synergy when tested in a cytotoxic assay

In an exemplary embodiment, synergy is assessed using the median-effect principle (Chou, et al., Adv Enzyme Regul 22:27-55 (1984)). This method is based on Michaelis-Menton kinetics and reduces combination effects to a numeric indicator, the combination index (C.I.). Where the combination index is less than 1, synergism is indicated. Where the combination index is equal to 1, summation is indicated. Where the combination index is greater than 1, antagonism is indicated. One skilled in the art will recognize that it is possible to see mixed effects over a range of C.I. values. Therefore, only combinations that are consistent over at least the majority of the drug concentration range are classified as synergistic, additive, or antagonistic.

In an exemplary embodiment, the combination index of an antineoplastic thiol-binding mitochondrial oxidant and a second antineoplastic agent is less than 1.0. In another exemplary embodiment, the combination index of an antineoplastic thiol-binding mitochondrial oxidant and a second antineoplastic agent is at least less than 0.9. In another exemplary embodiment, the combination index of an antineoplastic thiol-binding mitochondrial oxidant and a second antineoplastic agent is at least less than 0.8. In another exemplary embodiment, the combination index of an antineoplastic thiol-binding mitochondrial oxidant and a second antineoplastic agent is at least less than 0.7. In another exemplary embodiment, the combination index of an antineoplastic thiol-binding mitochondrial oxidant and a second antineoplastic agent is at least less than 0.6.

A number of biological assays are available to evaluate and to optimize the choice of specific combinations of compounds for optimal antitumor activity. These assays can be roughly split into two groups those involving in vitro exposure of agents to tumor cells and in vivo antitumor assays in rodent models and rarely, in larger animals. Both in vitro assay using tumor cells and in vivo assays in animal models are discussed below, and are equally applicable to determining whether an thiol-binding mitochondrial oxidant, a nucleic acid binding agent, or an antimetabolite base analog, exhibit antineoplastic properties.

Cytotoxic assays in vitro for a combination of an antineoplastic thiol-binding mitochondrial oxidant and a second antineoplastic agent generally involve the use of established tumor cell lines both of animal and, especially of human origin. These cell lines can be obtained from commercial sources such as the American Type Tissue Culture Laboratory in Bethesda, Md. and from tumor banks at research institutions. Exposures to combinations of the present invention may be carried out under simulated physiological conditions of temperature, oxygen and nutrient availability in the laboratory. The endpoints for these in vitro assays can involve: 1) colony formation; 2) a simple quantitation of cell division over time; 3) the uptake of so called “vital” dyes which are excluded from cells with an intact cytoplasmic membrane; 4) the incorporation of radiolabeled nutrients into a proliferating (viable) cell. Colony forming assays have been used both with established cell lines, as well as fresh tumor biopsies surgically removed from patients with cancer. In this type of assay, cells are typically grown in petri dishes on soft agar, and the number of colonies or groups of cells (>60 μ in size) are counted either visually, or with an automated image analysis system. A comparison is then made to the untreated control cells allowed to develop colonies under identical conditions. Because colony formation is one of the hallmarks of the cancer phenotype, only malignant cells will form colonies without adherence to a solid matrix. This can therefore be used as a screening procedure combinations of the present invention, and there are a number of publications which show that results obtained in colony forming assays correlates with clinical trial findings with the same drugs.

The enumeration of the total number of cells is a simplistic approach to in vitro testing with either cell lines or fresh tumor biopsies. In this assay, clumps of cells are typically disaggregated into single units which can then be counted either manually on a microscopic grid or using an automated flow system such as either flow cytometry or a Coulter® counter. Control (untreated) cell growth rates are then compared to the treated (with a combination of antineoplastic thiol-binding mitochondrial oxidant and a second antineoplastic agent) cell growth rates. Vital dye staining is another one of the older hallmarks of antitumor assays. In this type of approach cells, either untreated or treated with a cancer drug, are subsequently exposed to a dye such as methylene blue, which is normally excluded from intact (viable) cells. The number of cells taking up the dye (dead or dying) are the numerator with a denominator being the number of cells which exclude the dye. These are laborious assays which are not currently used extensively due to the time and the relatively non-specific nature of the endpoint.

In addition to vital dye staining, viability can be assessed using the incorporation of radiolabeled nutrients and/or nucleotides. This is the test method that was used in the Viking Lander to look for life on Mars with the endpoint being how much of a radioactive substance was taken up into a sample as evidence of life activity. In tumor cell assays, a typical experiment involves the incorporation of either (³H) tritium or ¹⁴C-labeled nucleotides such as thymidine. Control (untreated) cells are shown to take up a substantial amount of this normal DNA building block per unit time, and the rate of incorporation is compared to that in the drug treated cells. This is a rapid and easily quantitatable assay that has the additional advantage of working well for cells that may not form large (countable) colonies. Drawbacks include the use of radioisotopes which present handling and disposal concerns.

There are large banks of human and rodent tumor cell lines that are available for these types of assays. The current test system used by the National Cancer Institute uses a bank of over 60 established sensitive and multidrug-resistant human cells lines of a variety of cell subtypes. This typically involves 5-6 established and well-characterized human tumor cells of a particular subtype, such as non-small cell or small cell lung cancer, for testing new agents. Using a graphic analysis system called Compare®, the overall sensitivity in terms of dye uptake (either sulforhodamine B or MTT tetrazolium dye) are utilized. The specific goal of this approach is to identify combinations that are uniquely active in a single histologic subtype of human cancer. In addition, there are a few sublines of human cancer that demonstrate resistance to multiple agents and are known to, in some cases, express the multidrug resistance pump, p-glycoprotein. Assays using these resistant cells are currently underway for screening compounds both from NCI laboratories as well as any submitted from universities or private parties. The endpoint for the NCI assay is the incorporation of a protein dye called sulforhodamine B (for adherent tumor cells) and the reduction of a tetrazolium (blue) dye in active mitochondrial enzymes (for non-adherent, freely-floating types of cells). This latter method is particularly useful for hematologic cancers including myelomas, leukemias and lymphomas.

Generally, once a combination has demonstrated some degree of activity in vitro at inhibiting tumor cell growth, such as colony formation or dye uptake, antitumor efficacy experiments are performed in vivo. Rodent systems are almost exclusively used for initial assays of antitumor activity since tumor growth rates and survival endpoints are well-defined, and since these animals generally reflect the same types of toxicity and drug metabolism patterns as in humans. For this work, syngeneic (same gene line) tumors are typically harvested from donor animals, disaggregated, counted and then injected back into syngeneic (same strain) host mice. Anticancer combinations are typically then injected at some later time point(s), either by intraperitoneal, intravenous or administered by the oral routes, and tumor growth rates and/or survival are determined, compared to untreated controls or controls having only an antineoplastic thiol-binding mitochondrial oxidant or a second antineoplastic agent. In these assays, growth rates are typically measured for tumors injected growing in the front flank of the animal, wherein perpendicular diameters of tumor width are translated into an estimate of total tumor mass or volume. The time to reach a predetermined mass is then compared to the time required for equal tumor growth in the untreated control animals. In some embodiments, significant findings generally involve a >25% increase in the time to reach the predetermined mass in the treated animals compared to the controls. In other embodiments, significant findings involve a >42% increase in the time to reach the predetermined mass in the treated animals compared to the controls. The significant findings are termed tumor growth inhibition. For non-localized tumors such as leukemia, survival can be used as an endpoint and a comparison is made between the treated animals and the untreated or solvent treated controls. In general, a significant increase in life span for a positive new agent is again >20-42% longer life span due to the treatment. Early deaths, those occurring before any of the untreated controls, generally indicate toxicity for a new compound.

For all these assays, the anticancer combinations are generally tested at doses very near the lethal dose and 10% (LD₁₀) and/or at the determined maximally-tolerated dose, that dose which produces significant toxicity, but no lethality in the same strain of animals and using the same route of administration and schedule of dosing. Similar studies can also be performed in rat tumor models although, because of the larger weight and difficulty handling these animals they are less preferred than the murine models.

More recently, human tumors have been successfully transplanted in a variety of immunologically deficient mouse models. In the initial work, a mouse called the nu/nu or “nude” mouse was used to develop in vivo assays of human tumor growth. In nude mice, which are typically hairless and lack a functional thymus gland, human tumors (millions of cells) are typically injected in the flank and tumor growth occurs slowly thereafter. This visible development of a palpable tumor mass is called a “take”. Anticancer drugs are then injected by some route (IV, IM, SQ, PO) distal to the tumor implant site, and growth rates are calculated by perpendicular measures of the widest tumor widths as described earlier. A number of human tumors are known to successfully “take” in the nude mouse model, even though these animals are more susceptible to intercurrent infections due to the underlying immunologic deficiency. An alternative mouse model for this work involves mice with a severe combined immunodeficiency disease (SCID) wherein there is a defect in maturation of lymphocytes. Because of this, SCID mice do not produce functional B- and T-lymphocytes. However, these animals do have normal cytotoxic T-killer cell activity. Nonetheless, SCID mice will “take” a large number of human tumors. Animals with the SCID phenotype are screened for “leakiness” by measuring serum immunoglobulin production which should be minimal to undetectable if the SCID phenotype is maintained. Tumor measurements and drug dosing are generally performed as above. The use of SCID mice has in many cases displaced the nude mouse since SCID mice seem to have a greater ability to take a larger number of human tumors and are more robust in terms of lack of sensitivity to intercurrent infections. Again, positive compounds in the SCID mouse model are those that inhibit tumor growth rate by >20-42% compared to the untreated control.

Testing for drug resistance can involve any of the in vitro and in vivo models, although the in vitro models are better characterized. In these tests, a cell subline is developed for resistance to a particular agent generally by serial exposure to increasing concentrations of the anticancer combination either in vitro or rarely in vivo. Once a high degree of resistance is demonstrated (generally >4- to 5-fold) to a particular agent the cell line is further studied for mechanisms of resistance such as the expression of multidrug resistance membrane pumps such as p-glycoprotein or others. These resistant cell lines can then be tested for cross-resistance with classic anticancer agents to develop a response pattern for a particular cell line. Using this cell line one can then evaluate a new agent for its potential to be active in the resistant cells. This has allowed for the demonstration of both mechanisms of drug resistance, as well as the identification of agents which might have utility in human cancers that have become resistant to existing chemotherapy agents. More recently, the use of resistant human tumor cells has been extended to the SCID mouse model with the development of an in vivo model of multidrug-resistant human multiple myeloma.

All of these test systems are generally combined in a serial order, moving from in vitro to in vivo, to characterize the antitumor activity of an anticancer combination. In general, one wishes to find out what tumor types are particularly sensitive to a combination and conversely what tumor types are intrinsically resistant to a combination in vitro. Using this information, experiments are then planned in rodent models to evaluate whether or not the combinations that have shown activity in vitro will be tolerated and active in animals. The initial experiments in animals generally involve toxicity testing to determine a tolerable dose schedule and then using that dose schedule, to evaluate antitumor efficacy as described above. Active combinations from these two types of assays may then be tested in human tumors growing in SCID or nude mice and if activity is confirmed, these combinations then become candidates for potential clinical drug development.

IV. Assays for Measuring Characteristics of Antineoplastic Thiol-Binding Mitochondrial Oxidants

As described above, antineoplastic thiol-binding mitochondrial oxidants of the present invention are those compounds that inhibit or prevent the growth of cancer, are capable of binding thiol moieties, and promote oxidative stress and disruption of cellular mitochondrial membrane potential. In some embodiments, the antineoplastic thiol-binding mitochondrial oxidant inhibits or reduces activity of a ribonucleotide reductase inhibitor. Cytotoxic assays useful for determining whether a compound is antineoplastic are discussed above (see Assays for Testing the Anticancer Synergistic Activity of a Combination of an Antineoplastic Thiol-binding Mitochondrial Oxidant and a Second Antineoplastic Agent). Assays for measuring other characteristics are described below.

A. Thiol Binding Assays

The ability of a test compound to bind to a thiol-containing molecule may be assessed by mixing the test compound in aqueous solution with a thiol-containing molecule, such as cysteine or glutathione. The solution is incubated for sufficient time to allow binding of the thiol moiety to the test compound to form a reaction product. After incubating the mixture for a sufficient time, any appropriate separation method (e.g. thin layer chromatography (TLC)) may be performed on the solution to isolate the reaction product. After isolation, the reaction product is optionally further purified (e.g. filtration) and detected using any appropriate technique, such as nuclear magnetic resonance or mass spectroscopy.

Selection of the appropriate reaction times, reaction solvents, and elution solvents is well within the skill of those practiced in the chemical and biochemical arts. A more detailed discussion of thiol binding assays are provided in Iyengar et al., J. Med. Chem. 47: 218-223 (2004).

B. Oxidative Stress and Mitochondrial Membrane Potential Assays

The presence of oxidative stress may be assessed using an antibody capable of binding to oxidized nucleotides, such as the well characterized monoclonal antibody 8-OHdG. The appropriate cell line, such as myeloma cells, may be treated with a test compound at various time points. The cells may then be fixed with formaldehyde and subsequently permeabilized with methanol. The cell can then be immunostained with the appropriate anti-oxidized nucleotide antibody and visualized using any appropriate detection technique, such as a secondary antibody system (e.g. biotinylated secondary antibody and subsequent addition of Cy5-conjugated streptavidin). Nuclear localization may then be accomplished using an appropriate nuclear stain, such as YOYO-1® stain (Molecular Probes). Laser confocal microscopy may then be used to visualize oxidative damage within the mitochondrial cellular compartment.

Loss of the mitochondrial membrane potential (“MMP”) may be measured by flow cytometry based on the uptake of and retention of cationic charged dyes into undamaged mitochondria. Examples of useful dyes include MitoTracker Red®, also known as CMX-Ros, and JC-1 (both available from Molecular Probes, Eugene Oreg.). The dyes may passively diffuse across plasma membranes and taken up and preferentially retained in mitochondria with undamaged membranes which retain the electronegative inner membrane environment. As the MMP decreases, the dye signal intensity is reduced compared to undamaged mitochondria in control cells. The JC-1 reagent undergoes a fluorescent emission shift from red to green when the mitochondrial interior is depolarized after the MMP is lost. For a more detailed discussion of MMP assays, see Decaudin et al., Cytometry 25:333-340 (1996); and Manzini et al., J Cell Biol 138: 449-469 (1997).

Further details on assays for measuring oxidative stress and mitochondrial membrane potential may be found in Dvorakova et al., Neoplasia 97: 3544-3551(2001), Dvorakova et al., Biochemical Pharmacology 60: 749-758 (2000), Dvorakova et al., Anti-Cancer Drugs 13: 1031-1042 (2002), and Dvorakova et al., Molecular Cancer Therapeutics 1: 185-195 (2002).

C. Ribonucleotide Reductase Activity Assays

Ribonucleotide reductase (“RNR”) activity may be measured by first contacting a cell culture with the appropriate test compound. The cells are then harvested and the cell lysate purified by an appropriate technique to separate deoxycytidine (the specific product of RNR activity) and cytidine after phosphorylation (such as Affigel 601 column or a high-resolution HPLC C-18 column). The amount of deoxycytine product is measured and compared to the amount of product produced by the cell in the absence of added test compound thereby determining the ability of the test compound to inhibit or decrease RNR activity.

In an alternative method, deoxyribonucleotides (the product of RNR activity) are detected via coupling to the DNA polymerase reaction with enhanced detection using RNAse to degrade endogenous RNA.

For a more detailed discussion of RNR activity assays, see Wright et al., Adv Enzyme Regul 19:105-127 (1981); and Jong et al., J Biomed Sci 5:62-68 (1998).

V. Dosage

A pharmaceutical composition of the present invention can be micronized or powdered so that it is more easily dispersed and solubilized by the body. Processes for grinding or pulverizing drugs are well known in the art, for example, by using a hammer mill or similar milling device.

Dosage forms (compositions) suitable for internal administration contain from about 1.0 milligram to about 5000 milligrams of active ingredient per unit. In these pharmaceutical compositions, the active ingredient may be present in an amount of about 0.5 to about 95% by weight based on the total weight of the composition. Another convention for denoting the dosage form is in mg per meter squared (mg/m²) of body surface area (BSA). Typically, an adult will have approximately 1.75 m² of BSA. Based on the body weight of the patient, the dosage may be administered in one or more doses several times per day or per week. Multiple dosage units may be required to achieve a therapeutically effective amount. For example, if the dosage form is 1000 mg, and the patient weighs 40 kg, one tablet or capsule will provide a dose of 25 mg per kg for that patient. It will provide a dose of only 12.5 mg/kg for a 80 kg patient.

By way of general guidance, for humans a dosage of as little as about 1 milligrams (mg) per kilogram (kg) of body weight and up to about 10000 mg per kg of body weight is suitable as a therapeutically effective dose. Preferably, from about 5 mg/kg to about 2500 mg/kg of body weight is used. Other preferred doses range between 25 mg/kg to about 1000 mg/kg of body weight. However, a dosage of between about 2 milligrams (mg) per kilogram (kg) of body weight to about 400 mg per kg of body weight is also suitable for treating some cancers.

Intravenously, the most preferred rates of administration can range from about 1 to about 1000 mg/kg/minute during a constant rate infusion. A pharmaceutical composition of the present invention can be administered in a single daily dose, or the total daily dosage may be administered in divided doses of two, three, or four times daily. An antineoplastic thiol-binding mitochondrial oxidant is generally given in one or more doses on a daily basis or from one to three times a week.

A pharmaceutical composition of the present invention is administered by any conventional means available for use in conjunction with pharmaceuticals, either as individual therapeutic agents or in combination with other therapeutic agents.

The amount and identity of an antineoplastic thiol-binding mitochondrial oxidant and second antineoplastic agent in treating cancers, respectively, can vary according to patient response and physiology, type and severity of side effects, the disease being treated, the preferred dosing regimen, patient prognosis or other such factors.

The ratio of an antineoplastic thiol-binding mitochondrial oxidant to the second antineoplastic agent can be varied as needed according to the desired therapeutic effect, the observed side-effects of the combination, or other such considerations known to those of ordinary skill in the medical arts. Generally, the ratio of an antineoplastic thiol-binding mitochondrial oxidant to second antineoplastic agent can range from about 0.5%:99.5% to about 99.5%:0.5% on a weight basis. In an exemplary embodiment, the ratio range from about 20%:80% to about 80%:20%. In another exemplary embodiment, the ratio range from about 40%:60% to about 60%:40%. In another exemplary embodiment, the ratio range from about 45%:55% to about 55%:45%. In another exemplary embodiment, the ratio range is about 50%:50%.

When an antineoplastic thiol-binding mitochondrial oxidant is administered before or after second antineoplastic agent, the respective doses and the dosing regimen of an antineoplastic thiol-binding mitochondrial oxidant and the second antineoplastic agent can vary. The adjunct or combination therapy can be sequential, that is the treatment with antineoplastic thiol-binding mitochondrial oxidant and then the second antineoplastic agent (or vice versa), or it can be concomitant treatment wherein the antineoplastic thiol-binding mitochondrial oxidant and second antineoplastic agent are administered substantially at the same time. The sequential therapy can be within a reasonable time after the administration of the antineoplastic thiol-binding mitochondrial oxidant before administering the antineoplastic agent. The treatment with both agents at the same time can be in the same daily dose or in separate doses.

The exact regimen will depend on the disease being treated, the severity of the disease and the response to the treatment. For example, a full dosing regimen of an antineoplastic thiol-binding mitochondrial oxidant can be administered either before or after a full dosing regimen of the second antineoplastic agent, or alternating doses of an antineoplastic thiol-binding mitochondrial oxidant and the second antineoplastic agent can be administered. As a further example, an antineoplastic thiol-binding mitochondrial oxidant can be administered concomitantly with the second antineoplastic agent.

The identity of the second antineoplastic agent, the pharmaceutical carrier and the amount of an antineoplastic thiol-binding mitochondrial oxidant administered can vary widely depending on the species and body weight of mammal and the type of cancer or viral infections being treated. The dosage administered can vary depending upon known factors, such as the pharmacodynamic characteristics of a specific second antineoplastic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

An antineoplastic thiol-binding mitochondrial oxidant and the second antineoplastic agent can be administered together in a single dosage form or separately in two or more different dosage forms. These can be administered independently by the same route or by two or more different routes of administration depending on the dosage forms employed.

Suitable pharmaceutical compositions and dosage forms will preferably comprise an antineoplastic thiol-binding mitochondrial oxidant and optionally an anticancer agent or an antiviral compound. The ratio of an antineoplastic thiol-binding mitochondrial oxidant to anticancer agent or antiviral compound can range from about 1:0.01 to 10:1, and preferably 1:0.05 to 1:1 on a weight basis.

The dose and the range of anticancer agent or antiviral compound will depend on the particular agent or compound and the type of cancer or viral infection being treated. One skilled in the art will be able to ascertain the appropriate dose.

VI. Dosage Form

A dosage unit can comprise a single compound or mixtures of an antineoplastic thiol-binding mitochondrial oxidant with one or more second antineoplastic agents. An antineoplastic thiol-binding mitochondrial oxidant can be administered in oral dosage forms such as tablets, capsules, pills, powders, granules, elixirs, tinctures, suspensions, syrups, and emulsions. An antineoplastic thiol-binding mitochondrial oxidant or second antineoplastic agent can also be administered in intravenous (bolus or infusion), intraperitoneal, subcutaneous, or intramuscular form, all using dosage forms well known to those of ordinary skill in the pharmaceutical arts.

An antineoplastic thiol-binding mitochondrial oxidant or second antineoplastic agent is typically administered in admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. The unit will be in a form suitable for oral, rectal, topical, intravenous injection or parenteral administration.

The pharmaceutical compositions can be administered alone or it can be mixed with a pharmaceutically acceptable carrier. This carrier can be a solid or liquid, and the type of carrier is generally chosen based on the type of administration being used.

Specific examples of pharmaceutical acceptable carriers and excipients that can be used to formulate oral dosage forms of the present invention are well known to one skilled in the art. See, for example, U.S. Pat. No. 3,903,297, which is incorporated herein by reference in its entirety for all purposes. Techniques and compositions for making dosage forms useful in the present invention are also well known to one skilled in the art. See, for example, 7 Modern Pharmaceutics, Chapters 9 and 10 (Banker & Rhodes, Eds., 1979); Pharmaceutical Dosage Forms: Tablets (Lieberman et al., 1981); Ansel, Introduction to Pharmaceutical Dosage Forms 2^{nd} Ed. (1976); Remington's Pharmaceutical Sciences, ₁₇^{th} ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), all of which are incorporated herein by reference in their entirety for all purposes.

Tablets can contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Pharmaceutical compositions can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Pharmaceutical compositions can also be coupled to soluble polymers as targetable drug carriers or as a prodrug. Suitable soluble polymers include polyvinylpyrrolidone, pyran copolymer, polyhydroxylpropylmethacrylamide-phenol, polyhydroxyethylasparta-midephenol, and polyethyleneoxide-polylysine substituted with palmitoyl residues. Furthermore, an antineoplastic thiol-binding mitochondrial oxidant can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules can contain the active ingredient and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Pharmaceutical compositions can also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms can also include minerals and other materials to make them compatible with the type of injection or delivery system chosen.

Useful pharmaceutical dosage forms for administration of an antineoplastic thiol-binding mitochondrial oxidant are illustrated as follows:

A. Capsules

A large number of unit capsules are prepared by filling standard two-piece hard gelatin capsules each with 10 to 500 milligrams of powdered active ingredient, 5 to 150 milligrams of lactose, 5 to 50 milligrams of cellulose, and 6 milligrams magnesium stearate.

B. Soft Gelatin Capsules

A mixture of active ingredient in a digestible oil such as soybean oil, cottonseed oil or olive oil is prepared and injected by means of a positive displacement pump into gelatin to form soft gelatin capsules containing 100-500 milligrams of the active ingredient. The capsules are washed and dried.

C. Tablets

A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

D. Injectable Solution

A parenteral composition suitable for administration by injection is prepared by stirring 1.5% by weight of active ingredient in 10% by volume propylene glycol and water. The solution is made isotonic with sodium chloride and sterilized.

E. Suspension

An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

F. Kits

The present invention also includes pharmaceutical kits useful, for example, for the treatment of cancer, which comprise one or more containers containing a pharmaceutical composition comprising a therapeutically effective amount of an antineoplastic thiol-binding mitochondrial oxidant and a second antineoplastic agent, respectively. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. It should be understood that although the specified materials and conditions are important in practicing the invention, unspecified materials and conditions are not excluded so long as they do not prevent the benefits of the invention from being realized.

Pharmaceutical carriers can be a solid or liquid and the type is generally chosen based on the type of administration being used. The active agent can be coadministered in the form of a tablet or capsule, liposome, as an agglomerated powder or in a liquid form. Examples of suitable solid carriers include lactose, sucrose, gelatin and agar. Capsules or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

VII. Methods of Treatment

The method of treatment can be any suitable method that is effective in the treatment of the particular cancer or tumor type being treated. Treatment can be oral, rectal, topical, parenteral or intravenous administration or by injection into the tumor or cancer. The method of applying an effective amount also varies depending on the disorder or disease being treated. It is believed that parenteral treatment by intravenous, subcutaneous, or intramuscular application of an antineoplastic thiol-binding mitochondrial oxidant, formulated with an appropriate carrier, additional cancer inhibiting compound or compounds or diluent to facilitate application will be the preferred method of administering the compounds to warm blooded animals.

One skilled in the art will recognize that the efficacy of the compounds can be ascertained through routine screening using known cancer cell lines both in vitro and in vivo. Cell lines are available from American Tissue Type Culture or other laboratories.

The following examples are illustrative and not intended to be limiting of the invention.

A. Measuring Response to Pharmaceutical Formulations

Tumor load is assessed prior to therapy by means of objective scans of the tumor such as with x-ray radiographs, computerized tomography (CAT scans), nuclear magnetic resonance (NMR) scans or direct physical palpation of the tumor mass. Alternatively, the tumor may secrete a marker substance such as alphafetoprotein from colon cancer, CA125 antigen from ovarian cancer, or serum myeloma “M” protein from multiple myeloma. The levels of these secreted products then allow for an estimate of tumor burden to be calculated. These direct and indirect measures of the tumor load are done pretherapy, and are then repeated at intervals following the administration of the drug in order to gauge whether or not an objective response has been obtained. An objective response in cancer therapy generally indicates >50% shrinkage of the measurable tumor disease (a partial response), or complete disappearance of all measurable disease (a complete response). Typically these responses must be maintained for a certain time period, usually one month, to be classified as a true partial or complete response. In addition, there may be stabilization of the rapid growth of a tumor or there may be tumor shrinkage that is <50%, termed a minor response or stable disease. In general, increased survival is associated with obtaining a complete response to therapy and in some cases, a partial response if maintained for prolonged periods can also contribute to enhanced survival in the patient. Patients receiving chemotherapy are also typically “staged” as to the extent of their disease before and following chemotherapy are then restaged to see if this disease extent has changed. In some situations the tumor may shrink sufficiently and if no metastases are present, then surgical excision may be possible after chemotherapy treatment where it was not possible beforehand due to the widespread disease. In this case the chemotherapy treatment with the novel pharmaceutical compositions is being used as an adjuvant to potentially curative surgery. In addition, patients may have individual lesions in the spine or elsewhere that produce symptomatic problems such as pain and these may need to have local radiotherapy applied. This may be done in addition to the continued use of the systemic pharmaceutical compositions of the present invention.

B. Assessing Toxicity and Setting Dosing Regimens

Patients are assessed for toxicity with each course of chemotherapy, typically looking at effects on liver function enzymes and renal function enzymes such as creatinine clearance or BUN as well as effects on the bone marrow, typically a suppression of granulocytes important for fighting infection and/or a suppression of platelets important for hemostasis or stopping blood flow. For such myelosuppressive drugs, the nadir in these normal blood counts, is reached between 1-3 weeks after therapy and recovery then ensues over the next 1-2 weeks. Based on the recovery of normal white blood counts, treatments may then be resumed.

In general, complete and partial responses are associated with at least a 1-2 log reduction in the number of tumor cells (a 90-99% effective therapy). Patients with advanced cancer will typically have >10⁹ tumor cells at diagnosis, multiple treatments will be required in order to reduce tumor burden to a very low state and potentially obtain a cure of the disease.

C. Clinical Management of Patients

At the end of a treatment cycle with a novel pharmaceutical formulation which could comprise several weeks of continuous drug dosing, patients will be evaluated for response to therapy (complete and partial remissions), toxicity measured by blood work and general well-being classified performance status or quality of life analysis. The latter includes the general activity level of the patient and their ability to do normal daily functions. It has been found to be a strong predictor of response and some anticancer drugs may actually improve performance status and a general sense of well-being without causing a significant tumor shrinkage. The antimetabolite gemcitabine is an example of such a drug that was approved in pancreatic cancer for benefiting quality of life without changing overall survival or producing a high objective response rate. Thus, for some cancers that are not curable, the pharmaceutical formulations may similarly provide a significant benefit, well-being performance status, etc. without affecting true complete or partial remission of the disease.

In hematologic disorders such as multiple myeloma, lymphoma and leukemia, responses are not assessed via the measurement of tumor diameter since these diseases are widely metastatic throughout the lymphatic and hematogenous areas of the body. Thus, responses to these diffusely disseminated diseases are usually measured in terms of bone marrow biopsy results wherein the number of abnormal tumor cell blasts are quantitated and complete responses are indicated by the lack of detection (e.g. microscopic detection) of any tumor cells in a bone marrow biopsy specimen. With the B-cell neoplasm multiple myeloma a serum marker, the M protein, can be measured by electrophoresis and if substantially decreased this is evidence of the response of the primary tumor. Again, in multiple myeloma, bone marrow biopsies can be used to quantitate the number of abnormal tumor plasma cells present in the specimen. For these diseases generally higher dose therapy is typically used to affect responses in the bone marrow and/or lymphatic compartments.

The projected clinical uses for the novel pharmaceutical formulations are as treatments for: lung cancer, breast cancer, malignant melanoma, AIDS-related lymphoma, multidrug-resistant (MDR) tumors (Myeloma, Leukemia Breast and Colon Carcinoma), prostate cancer, multiple myeloma, a β-lymphocyte plasmacytoma, advanced stage ovarian epithelial cell cancer, metastatic melanoma, leukemias of lymphoid and nonlymphoid origin, metastatic colon cancer, breast cancers and metastatic lung cancers, and neoplasms of the endocrine and exocrine pancreas.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the synergistic combinations of the present invention are equally applicable to the methods of treating disease states and/or the pharmaceutical compositions described herein. All publications, patents, and patent applications cited herein are hereby incorporated by reference in their entirety for all purposes.

### EXAMPLES

The following examples are offered to illustrate, but not to limit the claimed invention.

Materials

Imexon was obtained as a generous donation from the National Cancer Institute and was manufactured by Seres Laboratories Incorporated (Santa Rosa, Calif.). Cisplatin was obtained from Bayer Corp (Spokane, Wash.). Cytarabine was purchased from Bedford Laboratories (Bedford, Ohio), dexamethasone was purchased from Sigma (St. Louis, Mo.), doxorubicin was obtained from Fujisawa USA (Deerfield, Ill.), and dacarbazine (DTIC) was purchased from Bayer Corp (West Haven, Conn.). 5-fluorouracil was purchased from Allergan Inc. (Irvine, Calif.), gemcitabine was purchased from Eli Lilly and Co. (Indiana, Ind.), melphalan and vinorelbine were obtained from GlaxoWellcome, Inc. (Research Triangle Park, N.C., and methotrexate was obtained from Bristol (Syracuse, N.Y.). Paclitaxel was purchased from Bristol (Princeton, N.J.), and taxotere was obtained from Aventis (Collegeville, Pa.).

Human malignant melanoma A375 cells and human myeloma 8226/s cells were obtained from the American Type Culture Collection (Rockville, Md.). Acute myelogenous leukemia (KG-1) cells were kindly provided by Dr. Alan List (University of Arizona, Tucson, Ariz.) and the pancreatic cancer cell line, MiaPaCa, was generously provided by Dr. Daniel Von Hoff (University of Arizona, Tucson, Ariz.). All cell lines were cultured in RPMI 1640 media (Gibco-BRL Products, Grand Island, N.Y.) enhanced with 10% (v/v) heat inactivated bovine calf serum (Hyclone Laboratories, Logan, Utah), 2 mM L-glutamine, penicillin (100 U/ml) and streptomycin (100 μg/ml) in a humidified incubator containing 5% CO₂ at 37° C.

Female SCID (c.B-17/lcrACC SCID) mice (5-6) weeks old were purchased from a breeding colony maintained by the University of Arizona Animal Care facility (Tucson, Ariz.) and housed according to the guidelines of the American Association for Laboratory Animal Care under protocols approved by the University of Arizona Institutional Animal Care and Use Committee. Mice were housed in standard micro-isolator caging on wood chip bedding and provided with Isoblox (Harlan/Teklad, Madison, Wis.). Mice received standard sterilized rodent chow (Harlan/Teklad, Madison, Wis.) and sterile water ad libitum while maintained on a 12 hour/12 hour light/dark schedule. The Institutional Animal Care and Use Committee for the University of Arizona approved all protocols. At the termination of the experiment, mice were euthanized according to procedures outlined by the American Veterinary Medical Association.

### Example 1

Example 1 illustrates a method of determining whether a combination of an antineoplastic thiol-binding mitochondrial oxidant and a second antineoplastic agent exhibits a synergistic cytotoxic effect in vitro.

96 well plates (BD Biosciences, Lexington, Ky.) were seeded with approximately 2500 cells in 160 μl of growth medium per well in the last eleven columns of each plate. The first column of each plate was filled with 160 μl of growth medium containing no cells to be used as a blank. After a 24-hour incubation period, the cells in the last ten columns were drugged (leaving row one as a blank and row two as a control with uninhibited cell growth) with either 40 μl imexon (an antineoplastic thiol-binding mitochondrial oxidant), 40 μl of a second antineoplastic agent, or 20 μl imexon and 20 μl of second antineoplastic agent. Twelve second antineoplastic agents were tested: cisplatin, cytarabine, dexamethasone, doxorubicin, dacarbazine (DTIC), 5-fluorouracil, gemcitabine, irinotecan, melphalan, methotrexate, paclitaxel, taxotere, and vinorelbine. The drug concentrations and ratios used in the combination studies were determined from the IC₅₀ values of single-drug experiments. The drug ranges used for each combination study were developed by making small concentration changes above and below the IC₅₀ value for each antitumor agent. The IC₅₀ of each second antineoplastic agent was compared to the IC₅₀ value for imexon to establish a fixed constant ratio that was used in the subsequent combination drug exposures. Five days after drugging the cells, 96-well plates containing 8226/s cells were analyzed using the MTT assay (Rubinstein, L. V. et al., J Natl Cancer Inst 82:1113-111 (1990)) while plates containing A375 cells were analyzed using the SRB assay (Skehan, P. et al. J Natl Cancer Inst 82:1107-1112 (1990)).

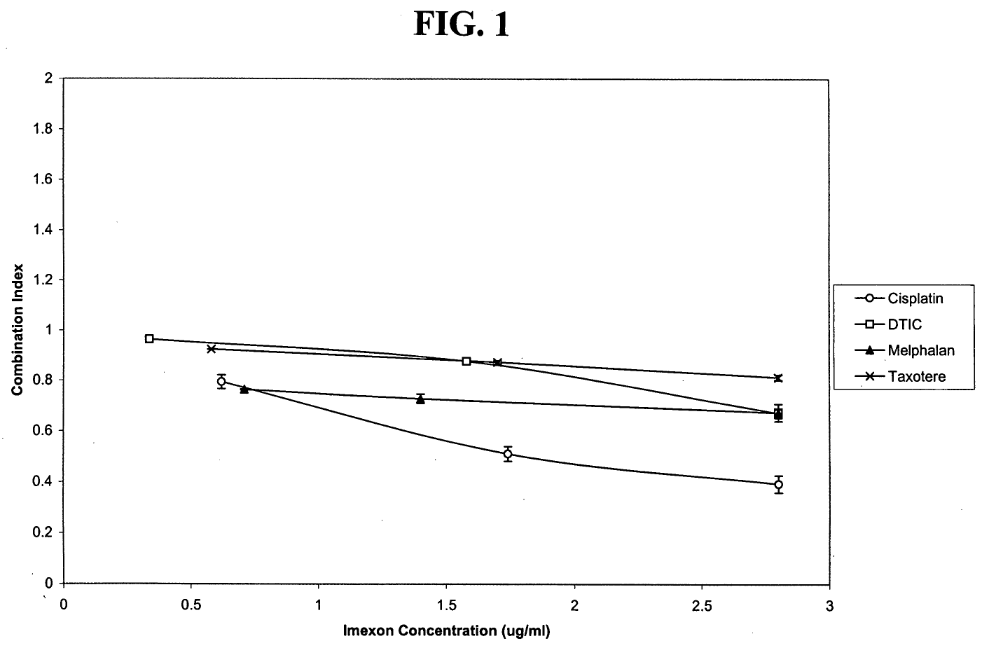

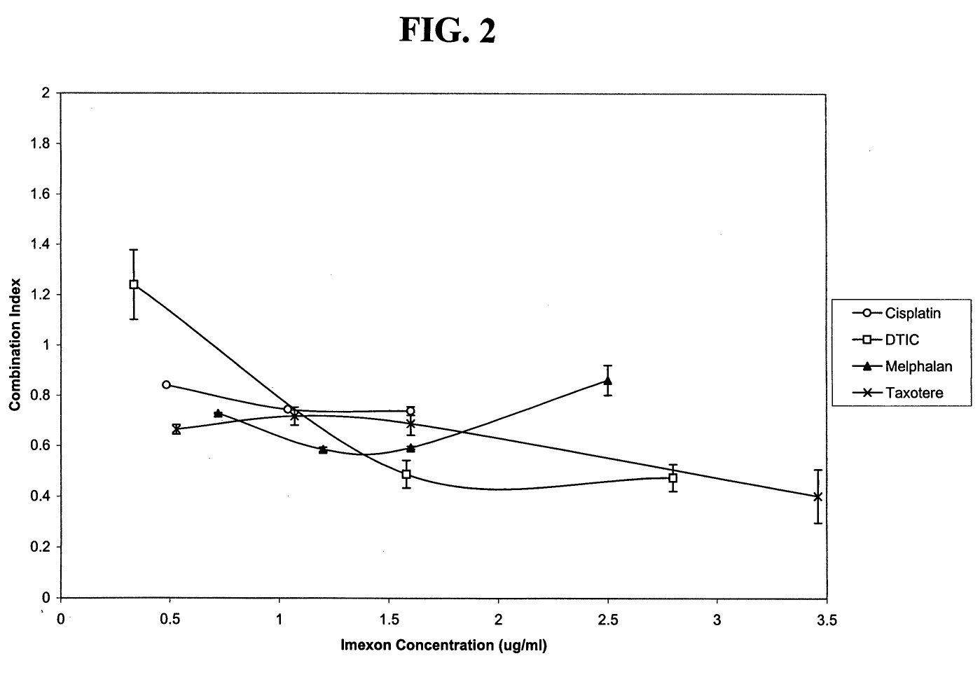

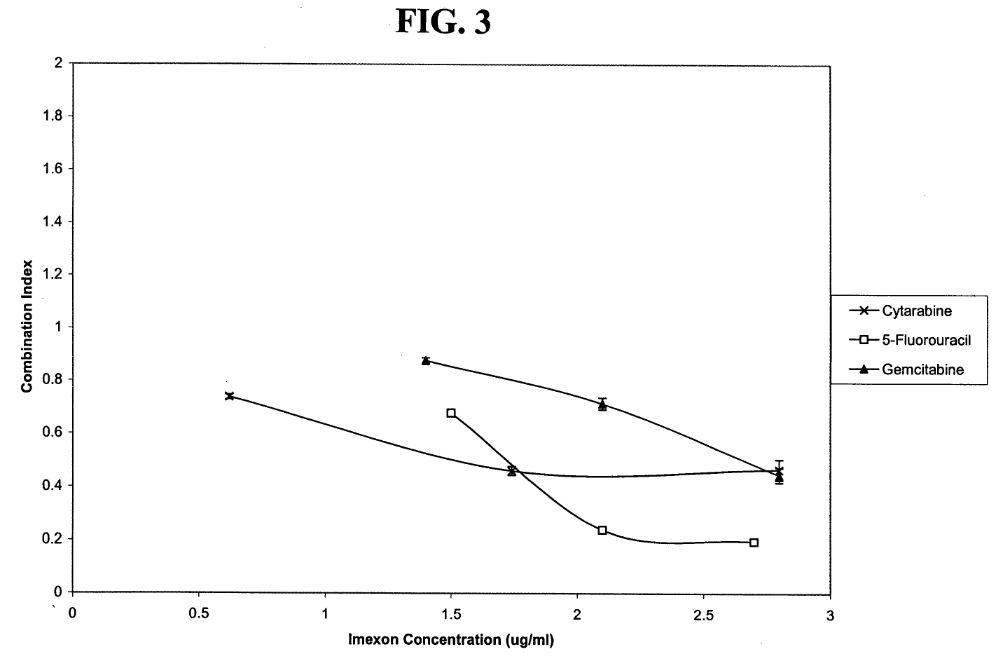

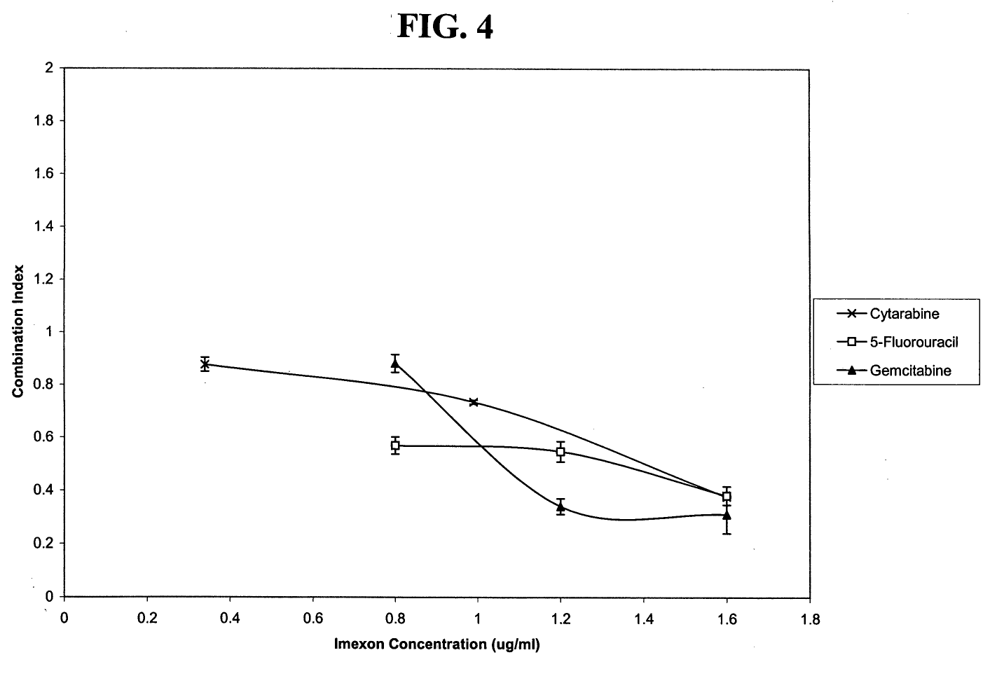

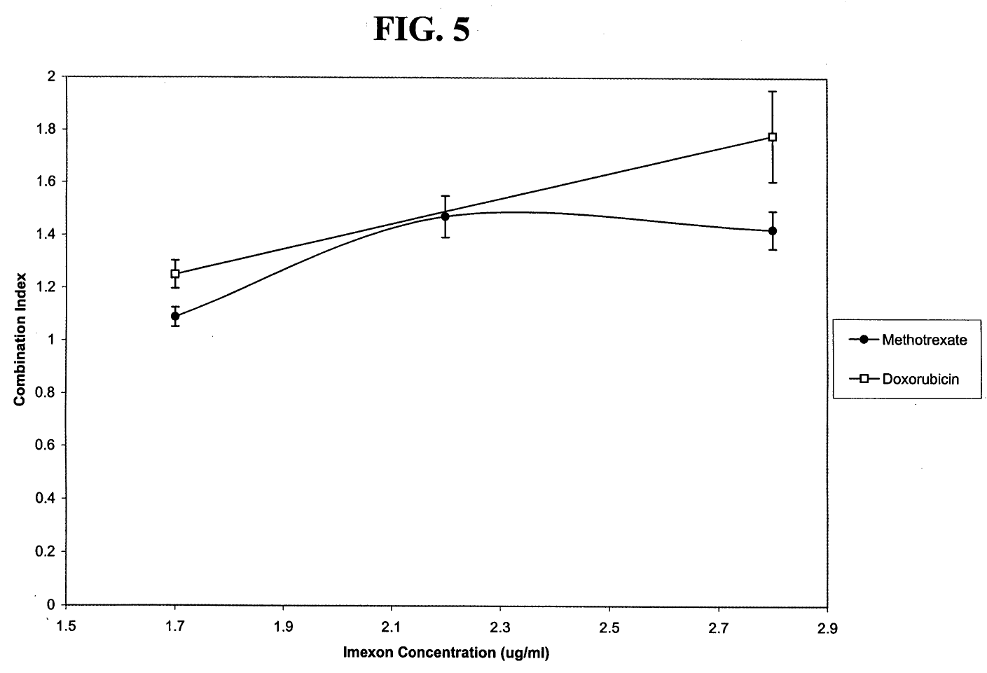

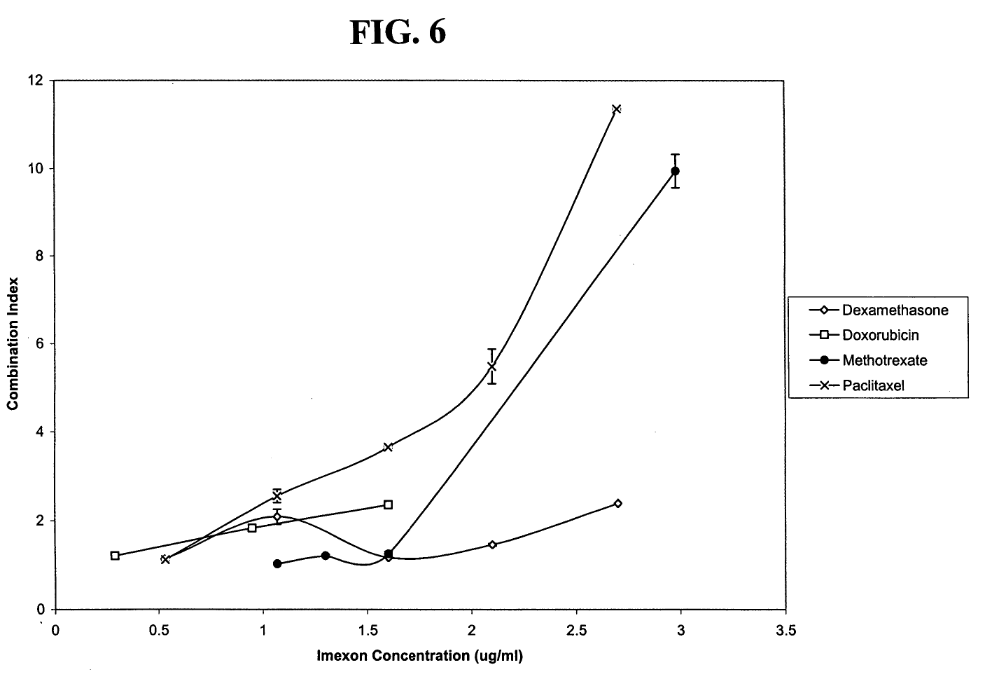

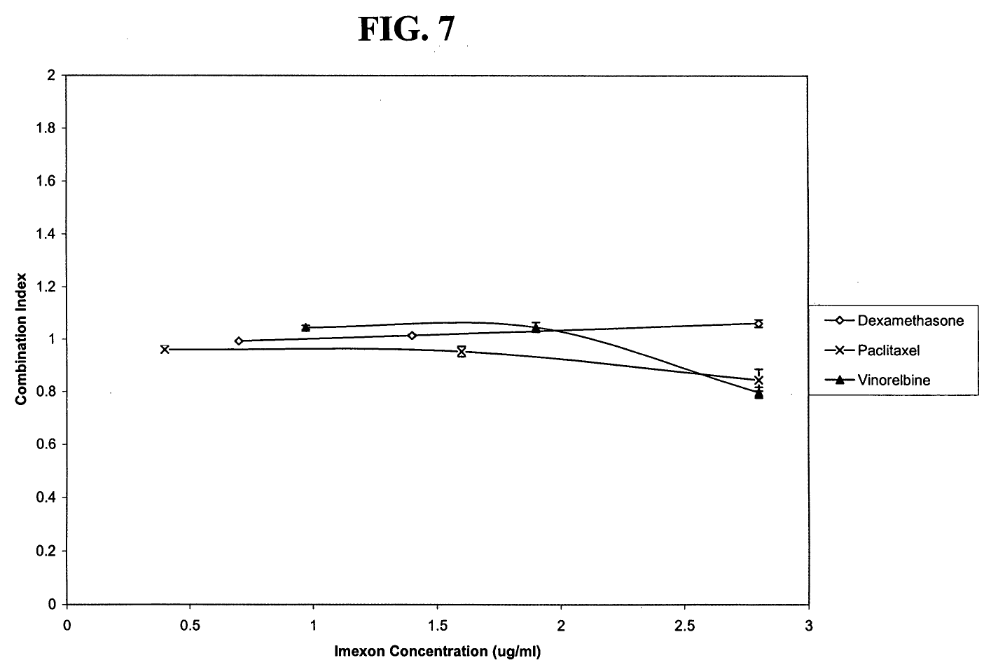

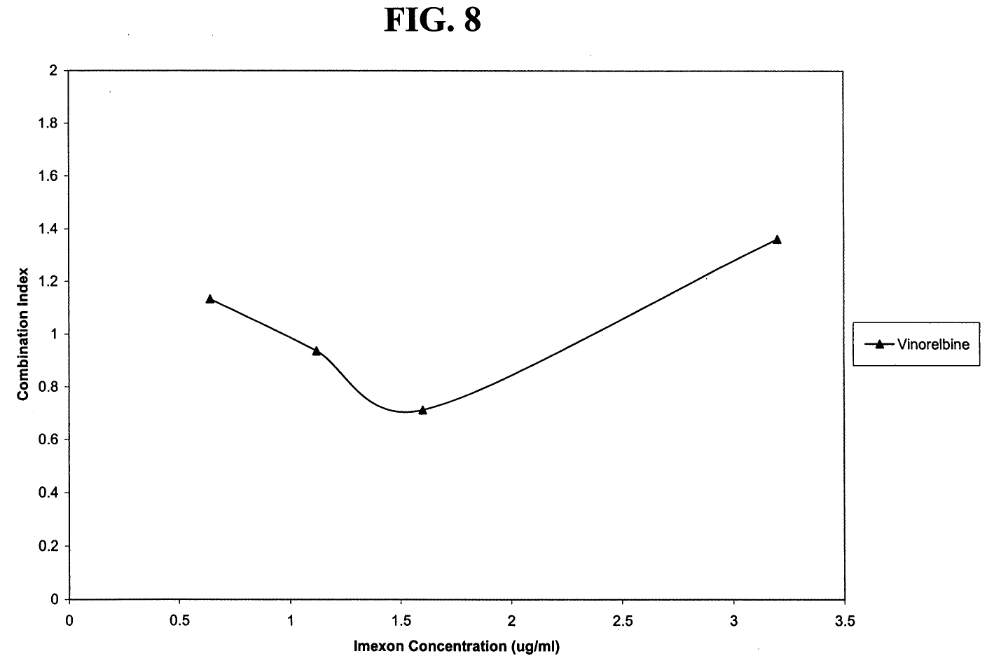

Synergy was determined from the combination index calculated according to the methods of Chou et al., Advances in Enzyme Regulation 22: 27-33 (1984). The combination indexes for the various combinations are shown as a function of imexon concentration in FIGS. 1-8 .

Table 1 below shows which of the second antineoplastic agents in combination with imexon demonstrated synergistic effects.

TABLE 1

| SECOND | | |
|---|---|---|
| ANTINEOPLASTIC | A375 CELL | |
| AGENT | LINE | 8226/S CELL LINE |
| cisplatin | Synergistic | Synergistic |
| cytarabine | Synergistic | Synergistic |
| dexamethasone | Additive | Antagonistic |
| doxorubicin | Antagonistic | Antagonistic |
| dacarbazine (DTIC) | Synergistic | Synergistic |
| 5-fluorouracil | Synergistic | Synergistic |
| gemcitabine | Synergistic | Synergistic |
| irinotecan | N/A | Antagonistic |
| melphalan | Synergistic | Synergistic |
| methotrexate | Antagonistic | Antagonistic |
| paclitaxel | Additive | Antagonistic |
| taxotere | Synergistic | Synergistic |
| vinorelbine | Additive | Additive |

### Example 2

Example 2 illustrates a method of determining whether a combination of an antineoplastic thiol-binding mitochondrial oxidant and a second antineoplastic agent exhibits a synergistic anticancer effect in vivo.

### Example 2.1

Pancreatic Cancer in SCID Mice

Gemcitabine and imexon were used in combination to treat pancreatic cancer in SCID mice. Sixteen SCID mice were inoculated with 10×10⁶ viable MiaPaCa tumor cells on day 0 by subcutaneous injection in the right rear flank. Four mice were used as controls and received no treatment. Another 4 mice were subsequently treated with imexon by a schedule of 100 mg/kg/day for 9 days beginning on day 1. A group of 4 mice receiving gemcitabine were treated at 180 mg/kg/day on days 1, 5, and 9. The final 4 mice received imexon at 100 mg/kg/day for 9 days and gemcitabine at 180 mg/kg/day on days 1, 5, and 9.

Tumor growth was measured in millimeters weekly using calipers to determine length and width. Mouse weight and survival were also monitored weekly. Tumor volume was calculated using the formula:
(length×width²)/2

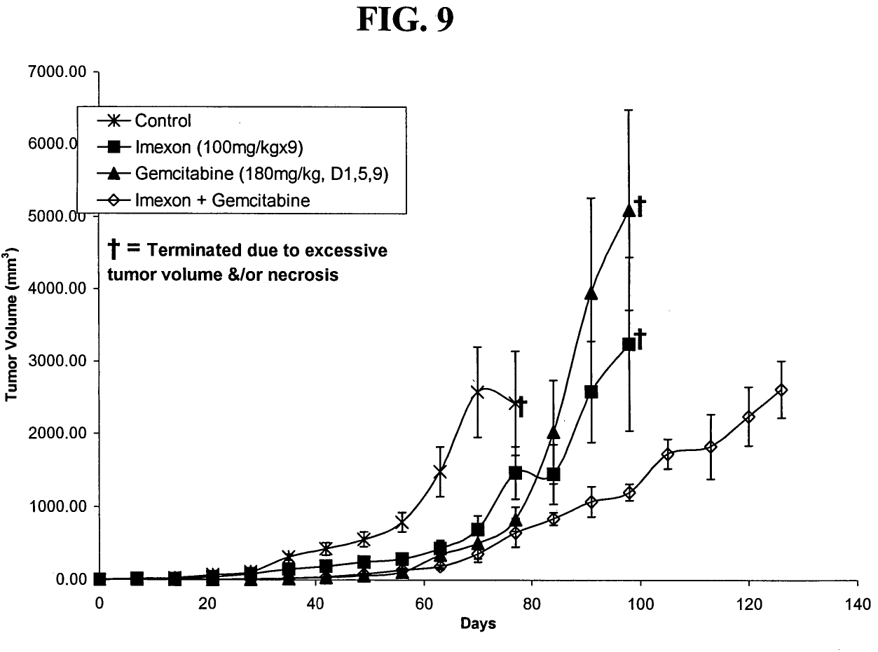

As shown in FIG. 9, the SCID mice treated with a combination of gemcitabine and imexon demonstrated a higher degree of tumor growth inhibition than the control mice, imexon-treated mice, and gemcitabine-treated mice

### Example 2.2

Myeloid Leukemia in SCID Mice

Cytarabine and imexon were used in combination to treat human KG-1 acute myeloid leukemia in SCID mice. Twenty SCID mice were inoculated with 10×10⁶ viable KG-1 leukemia cells on day 0 by subcutaneous injection in the right rear flank. Four mice were used as controls and received no treatment. A group of 4 mice were treated with imexon by a schedule of 100 mg/kg/day for nine days beginning on day 1. Another group of 4 mice received imexon at 150 mg/kg/day for five days beginning on day 1. Four mice were treated with cytarabine at 800 mg/kg/day on days 1, 5, and 9. The final group was treated with a combination of the two drugs, receiving imexon at 100 mg/kg/day for nine days and cytarabine at 800 mg/kg/day on days 1, 5, and 9.

Tumor growth was measured in millimeters weekly using calipers to determine length and width. Mouse weight and survival were also monitored weekly. Tumor volume was calculated using the formula:
(length×width²)/2

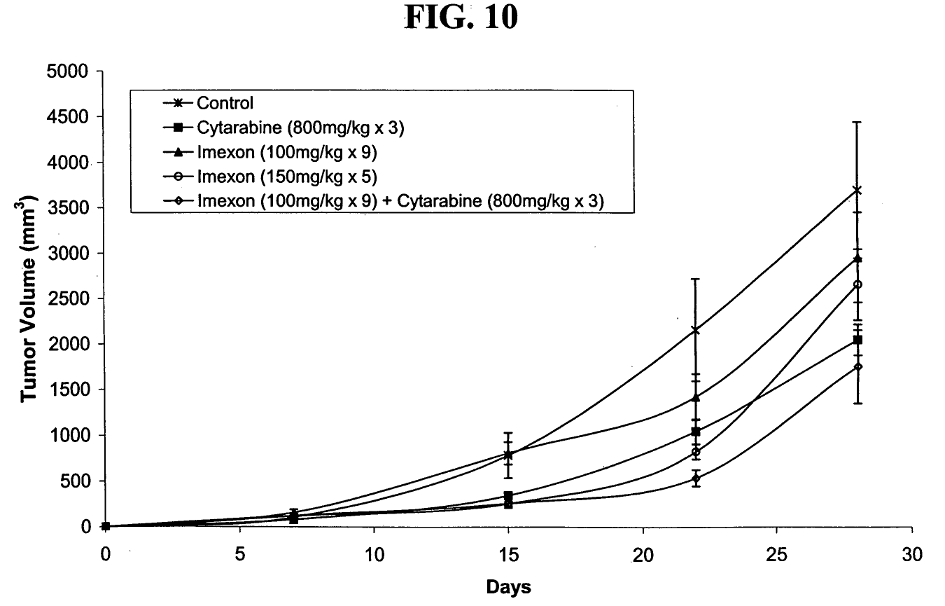

As shown in FIG. 10, the combination of cytarabine and imexon showed a greater extent of tumor growth inhibition than either concentration of imexon-treated mice, cytarabine-treated mice, or the control group.

### Example 3

Example 3 shows toxicology results from an experiment in which imexon and a second antineoplastic agent is administered to mice.

A toxicology study was performed in non-tumor bearing (i.e., normal) mice given imexon (100 mg/kg/day×9 days) with either gemcitabine (180 mg/kg days 1, 5 and 9) or cytarabine (800 mg/kg days 1, 4 and 7). The tests were conducted to evaluate whether there was increased bone marrow toxicity or decreased renal and hepatic function for imexon combined with either agent. The results of the platelet counts for mice treated with imexon and cytarabine or gemcitabine are shown below in Table 2.

| | | Treat- | Mean Platelet Counts | | |
|---|---|---|---|---|---|
| | Doses | ment | (SD) × 1000/μL | | |
| Agents | (mg/Kg) | Day | Day 8 | Day 10 | Day 12 |
| Imexon | 100 | 1-9 | — | 880 (180) | 920 (138) |
| Cytarabine | 800 | 1, 4 | 1039 (97) | 919 (107) | |
| | | and 7 | | | |
| Gemcitabine | 180 | 1, 5 | — | 777 | 678 (111) |
| | | and 9 | | | |
| Imexon + | 100 + 800 | | 724 (145) | 605 (236) | 681 (234) |
| Cytarabine | | | | | |
| Imexon + | 100 + 180 | | — | 454 (184) | 676 (397) |
| Gemicitabine | | | | | |

The results show that there were no significant effects on renal or liver function for the combination. There was a decrease in the white blood count for each combination, but the levels did not reach the lower limit for the normal range of WBC values. Almost all of the decrease involved the lymphocytes. There were no effects on neutrophils, which are believed to be the main targeted normal bone marrow cells in humans. The number of red blood cells increased slightly with imexon. Similarly, the platelet counts dropped with each combination, but not to significantly low levels. Overall no significant bone marrow toxicity was observed at full dose combinations of imexon with cytarabine or gemcitabine.

1. A method for treating cancer in a human patient in need of such treatment, said method comprising administering to the patient a therapeutically effective amount of a composition comprising an antineoplastic thiol-binding mitochondrial oxidant and an antineoplastic nucleic acid binding agent, said amount providing a synergistic therapeutic cytotoxic effect.

2. The method of claim 1, wherein said antineoplastic thiol-binding mitochondrial oxidant comprises an aziridine ring.

3. The method of claim 1, wherein said antineoplastic thiol-binding mitochondrial oxidant is a substituted or unsubstituted aziridine-1-carboxamide.

4. The method of claim 1, wherein said antineoplastic thiol-binding mitochondrial oxidant has the formula: 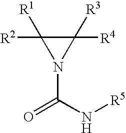
wherein
R¹, R², R³, R⁴ and R⁵ are independently selected from the group consisting of hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R⁴ and R⁵ are optionally joined together to form a substituted or unsubstituted 5 to 7 membered ring.

5. The method of claim 1, wherein said antineoplastic thiol-binding mitochondrial oxidant is imexon.

6. The method of claim 4, wherein R⁴ is cyano.

7. The method of claim 1, wherein said antineoplastic nucleic acid binding agent is an antineoplastic DNA binding agent.

8. The method of claim 1, wherein said antineoplastic nucleic acid binding agent is selected from the group consisting of nitrogen mustard, mitomycin derivative, alkyl sulfonate, nitroso urea, platinum complex, altretamine, and imidazole carboxamide.

9. The method of claim 1, wherein said antineoplastic nucleic acid binding agent is selected from the group consisting of nitrogen mustard, imidazole carboxamide, and platinum complex.

10. The method of claim 1, wherein said antineoplastic nucleic acid binding agent is selected from the group consisting of melphalan, cyclophosphamide, carmustine, mechlorethamine, thiotepa, chlorambucil, lomustine, ifosfamide, mitomycin C, cisplatin, carboplatin, oxaliplatin and dacarbazine.

11. The method of claim 1, wherein said cancer is selected from multiple myeloma, β-lymphocyte plasmacytoma, ovarian cancer, melanoma, leukemia, colon cancer, breast cancer, lung cancers, and pancreatic cancer.

12. The method of claim 11, wherein said pancreatic cancer is adenocarcinoma of the pancreas.

13. The method of claim 5, wherein said antineoplastic nucleic acid binding agent is not cyclophosphamide.

14. A method for treating cancer in a patient in need of such treatment, said method comprising administering to the patient a therapeutically effective amount of a composition comprising an antineoplastic thiol-binding mitochondrial oxidant and an antineoplastic antimetabolite base analog, said amount providing a synergistic therapeutic cytotoxic effect.

15. The method of claim 14, wherein said antineoplastic thiol-binding mitochondrial oxidant comprises an aziridine ring.

16. The method of claim 14, wherein said antineoplastic thiol-binding mitochondrial oxidant has the formula: 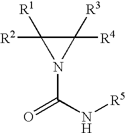
wherein
R¹, R², R³, R⁴ and R′ are independently selected from the group consisting of hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R⁴ and R⁵ are optionally joined to form a substituted or unsubstituted 5 to 7 membered ring.

17. The method of claim 14, wherein said antineoplastic thiol-binding mitochondrial oxidant is imexon.

18. The method of claim 16, wherein R⁴ is cyano.

19. The method of claim 14, wherein said antineoplastic antimetabolite base analog is selected from the group consisting of mercaptopurine, thioguanine, azathioprine, fludarabine, cladribine, pentostatin, fluorouracil, cytarabine, capecitabine, gemcitabine, and floxuridine.

20. The method of claim 14, wherein said antineoplastic antimetabolite base analog is selected from the group consisting of 5-fluorouracil, cytarabine, and gemcitabine.

21. The method of claim 14, wherein said antineoplastic antimetabolite base analog is gemcitabine.

22. The method of claim 14, wherein said cancer is selected from multiple myeloma, β-lymphocyte plasmacytoma, ovarian cancer, melanoma, leukemia, colon cancer, breast cancer, lung cancers, and pancreatic cancer.

23. The method of claim 22, wherein said pancreatic cancer is adenocarcinoma of the pancreas.

24. A method for treating cancer in a patient in need of such treatment, said method comprising administering to the patient a therapeutically effective amount of a composition comprising an antineoplastic thiol-binding mitochondrial oxidant and docetaxel, said amount providing a synergistic therapeutic cytotoxic effect.

25. The method of claim 24, wherein said antineoplastic thiol-binding mitochondrial oxidant comprises an aziridine ring.

26. The method of claim 24, wherein said antineoplastic thiol-binding mitochondrial oxidant has the formula: 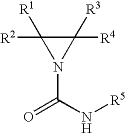
wherein
R¹, R², R³, R⁴ and R⁵ are independently selected from the group consisting of hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R⁴ and R⁵ are optionally joined to form a substituted or unsubstituted 5 to 7 membered ring.

27. The method of claim 26, wherein R⁴ is cyano.

28. The method of claim 24, wherein said antineoplastic thiol-binding mitochondrial oxidant is imexon.

29. The method of claim 24, wherein said cancer is selected from multiple myeloma, β-lymphocyte plasmacytoma, ovarian cancer, melanoma, leukemia, colon cancer, breast cancer, lung cancers, and pancreatic cancer.

30. The method of claim 24, wherein said pancreatic cancer is adenocarcinoma of the pancreas.
